(19) United States
(12) Patent Application Publication
(10) Pub. No.: US 2015/0052636 A1
(43) Pub. Date: Feb. 19, 2015

(54) Regulatory Nucleic Acid Molecules for Reliable Gene Expression in Plants

(71) Applicants: Julia Verena Hartig, Durham, NC (US); Maarten Hendrik Stuiver, Neustadt (DE); Josef Martin Kuhn, Limburgerhof (DE); Alrun Nora Burgmeier, Chapel Hill, NC (US)

(72) Inventors: Julia Verena Hartig, Durham, NC (US); Maarten Hendrik Stuiver, Neustadt (DE); Josef Martin Kuhn, Limburgerhof (DE); Alrun Nora Burgmeier, Chapel Hill, NC (US)

(73) Assignee: BASF Plant Science Company GmbH, Ludwigshafen (DE)

(21) Appl. No.: 14/344,955

(22) PCT Filed: Sep. 4, 2012
(86) PCT No.: PCT/IB2012/054549

§ 371 Date: Nov. 4, 2014

Related U.S. Application Data
(60) Provisional application No. 61/627,037, filed on Sep. 16, 2011.

(30) Foreign Application Priority Data
Sep. 15, 2011 (EP) 11181420.8

(51) Int. Cl. C12N 15/82

(52) U.S. Cl. CPC C12N 15/8216
USPC 800/287; 800/278; 435/320.1; 435/419; 800/298; 435/252.3; 435/254.11; 435/254.2

(57) ABSTRACT

The invention relates to methods and means for enhancing the reliability of expression in transgenic plants by reducing the coefficient of variation of expression and/or the number of no- or low-expressing plants in a population of plants.

### DESCRIPTION OF THE INVENTION

Efficient expression of a transgene is not only dependent on an optimal set of promoters and terminators. In a certain percentage of events the transgene is transcriptionally silent. In extreme cases, e.g. in transgenic cereals, up to 50% of events from a specific transgene can be silenced over successive generations (Vain et al., 1998). Several approaches have been investigated to prevent this negative effect on transgene expression.

Scaffold/Matrix Attachment Regions (S/MARs) have been experimentally defined as DNA elements that can attach to isolated eukaryotic nuclear matrix in an in vitro assay. S/MARs are at least 300 bp long but often exceed lengths of 1000 bp. Most S/MARs have an NT-content of 65% to more than 90%, though with little sequence conservation. They have been implicated as DNA domain-defining elements (Boulikas, 1995; Bode, 1995). A possible mode of action may be that they attach cis-regulatory elements (e.g. promoters and enhancers) to the nuclear matrix, where they become accessible to matrix-bound transcription factors (Cockerill et al., 1987; Bode et al. 2000). There have been reports that by positioning S/MARs upstream and downstream of a transgene, gene expression could be enhanced, possibly by blocking inhibitory position effects at the integration site (Lorence et al., 2004; Allen et al., 2005). Even though there are some reports where S/MARs had a positive effect on transgene expression in plants (Butaye et al., 2004, Oh et al., 2005, Torney et al., 2004, Xue et al., 2005), there is conflicting evidence concerning their effect on reducing variation between events (Liu and Tabe, 1998; Schoffel et al. 1993; Van der Greest and Hall, 1994; Mlynarova et al. 1995; Vain et al. 1999; De Bolle et al., 2007; Li et al, 2008).

Until today S/MARs remain highly elusive and cannot be reliably identified by bioinformatic means, with very little knowledge about associated effector proteins (De Bolle et al., 2007). Their length and their having to flank the transgene on both sides limit their usefulness in commercial plant transformation vectors. Moreover, the high NT-content makes normal methods of genetic manipulation difficult. According to the loop model of S/MAR regulation they exert influences on gene expression often over several thousand base-pairs.

Insulators are another type of DNA boundary element that has been reported to prevent position effects of surrounding chromatin on transgene expression. Interpretation of their mode of action varies between formation of individual chromatin domains that are able to block enhancer/promoter interaction to preventing the spread of heterochromatin into neighboring regions (Kuhn and Geyer, 2003; Gaszner and Felsenfeld, 2006).

Among the best studied insulators from animals are the gypsy and scs/scs′ insulators from Drosophila (Barolo at al., 2000; Markenstein et al., 2008) and the beta-globin HS4 insulator from chicken (Wang et al., 2009). There have been some successes in heterologously expressing animal insulators in plants to reduce the position effect on transgenes, leading to more reliable gene expression (Nagaya et al., 2001; She et al., 2010). However, the coefficient of variation could be reduced more pronouncedly (˜5-fold instead of ˜3-fold), if the heterologous insulator binding protein for the gypsy insulator was co-expressed (She et al., 2010). No efficient plant insulators have been identified so far. This is in part due to the fact that no sequence similarity can be found between known insulator elements (Sultana et al., 2011).

The necessity to include the insulator both upstream and downstream of the sequence intended for reliable expression and the need to co-express the heterologous binding protein limit the flexibility of use for commercial plant transformation.

Some authors have also pointed to a possible connection between both insulators and S/MARs: The insulator binding proteins CTCF in vertebrates and Su(Hw) in Drosophila have been reported to at least partially reside in the nuclear matrix (Byrd and Corces, 2003; Dunn et al., 2003; Yusufzai and Felsenfeld, 2004).

Introns have been reported in animals and various monocotyledonous (e.g. Callis et al., 1987; Vasil et al., 1989; Bruce et al., 1990; Lu et al., 2008) and dicotyledonous plants (e.g. Chung et al., 2006; Kim et al., 2006; Rose et al., 2008) to increase gene expression, also known as Intron Mediated Enhancement (IME). However, this current invention describes the novel influence of introns or nucleic acid molecules derived from or comprised in introns, on the reliability of gene expression. Reliable gene expression may be defined by a low percentage of non-expressers in the population of transgenic plants derived from transformation with one T-DNA construct. It follows that the absolute number of necessary events can be reduced since the probability to isolate plants exhibiting suitable expression is higher. Another definition of reliable gene expression is a reduced coefficient of variation of expression in a population of transgenic plants. Therefore the current invention has the ability to considerably reduce necessary time and costs for commercial plant transformation.

### DETAILED DESCRIPTION OF THE INVENTION

A first embodiment of the invention comprises a method for reducing the coefficient of variation of expression in a population of plants preferably independent plants, for example independent primary transformant plants comprising the steps of
a) providing a recombinant nucleic acid construct comprising at least one Reliability Enhancing Nucleic Acid (RENA) molecule selected from the group comprising
   i) the nucleic acid molecule having a sequence as defined in any of SEQ ID NO: 1 to 16 or 94 to 116666 or
   ii) a nucleic acid molecule having a sequence with an identity of 80% or more to any of the sequences as defined by SEQ ID NO:1 to 16 or 94 to 116666, preferably, the identity is 85% or more, more preferably the identity is 90% or more, even more preferably, the identity is 95% or more, 96% or more, 97% or more, 98% or more or 99% or more, in the most preferred embodiment, the identity is 100% to any of the sequences as defined by SEQ ID NO:1 to 16 or 94 to 116666 or
   iii) a fragment of 100 or more consecutive bases, preferably 150 or more consecutive bases, more preferably 200 consecutive bases or more even more preferably 250 or more consecutive bases of a nucleic acid molecule of i) or ii) or
   iv) a nucleic acid molecule of 100 nucleotides or more, 150 nucleotides or more, 200 nucleotides or more or 250 nucleotides or more, hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. or 65° C., preferably 65° C. to a nucleic acid molecule comprising at least 50, preferably at least 100, more preferably at least 150, even more preferably at least 200, most preferably at least 250 consecutive nucleotides of a transcription enhancing nucleotide sequence described by SEQ ID NO:1 to 16 or 94 to 116666 or the complement thereof. Preferably, said nucleic acid molecule is hybridizing under conditions equivalent to hybridization in 7% sodium dodecyl sulfate (SDS), 0.5 M NaPO₄, 1 mM EDTA at 50° C. with washing in 1×SSC, 0.1% SDS at 50° C. or 65° C., preferably 65° C. to a nucleic acid molecule comprising at least 50, preferably at least 100, more preferably at least 150, even more preferably at least 200, most preferably at least 250 consecutive nucleotides of a transcription enhancing nucleotide sequence described by SEQ ID NO:1 to 16 or 94 to 116666 or the complement thereof, more preferably, said nucleic acid molecule is hybridizing under conditions equivalent to hybridization in 7% sodium dodecyl sulfate (SDS), 0.5 M NaPO₄, 1 mM EDTA at 50° C. with washing in 0.1×SSC, 0.1% SDS at 50° C. or 65° C., preferably 65° C. to a nucleic acid molecule comprising at least 50, preferably at least 100, more preferably at least 150, even more preferably at least 200, most preferably at least 250 consecutive nucleotides of a transcription enhancing nucleotide sequence described by any of the sequences as defined by SEQ ID NO:1 to 16 or 94 to 116666 or
   v) a nucleic acid molecule which is the complement or reverse complement of any of the previously mentioned nucleic acid molecules under i) to vi),
b) functionally linking the RENA molecule as defined under i) to v) to a promoter and/or a nucleic acid molecule of interest wherein at least the promoter is heterologous to at least one of the RENA molecules and
c) integrating the RENA molecule functionally linked to a promoter and/or a nucleic acid molecule of interest in the genome of plant cells, plants or part thereof and
d) regenerating plants, preferably independent primary transformant plants from the transgenic plant cells, plants or part thereof produced in step c),
wherein the molecules as defined under ii) to v) reduce the variation of expression by least 10%, preferably at least 20%, for example 30%, more preferably at least 40%, for example 50%, even more preferably 60%, 70% or 80%, even more preferably at least 90 or 95%, most preferably 100% of the corresponding nucleic acid molecule having the sequence of SEQ ID NO: 1 to 16 or 94 to 116666, and

wherein the population of plants exhibit an at least 10%, preferably at least 20%, for example 30%, more preferably at least 40%, for example 50%, even more preferably 60%, 70% or 80%, even more preferably at least 90% or 95%, most preferably 100% lower coefficient of variation of expression of the nucleic acid molecule of interest compared to a respective population comprising in their genome a respective recombinant nucleic acid construct not comprising the respective RENA molecule as defined in i) to v).

Preferably, the population of plants comprising the recombinant construct comprising the RENA molecule and the respective control population of plants are populations of independent primary transformant plants.

Preferably the RENA molecule is heterologous to both the promoter and the nucleic acid of interest to which it is functionally linked.

The coefficient of variation is defined as standard deviation of expression of the nucleic acid of interest within a population of plants, for example primary transformand plants, divided by the mean of expression of the respective nucleic acid of interest in the respective population of plants.

Reliable gene expression is defined by a low percentage of non- or low-expressers in the population of transgenic plants derived from transformation with one T-DNA comprising an expression construct comprising at least one RENA molecule functionally linked to a promoter and a nucleic acid of interest, wherein at least the promoter is heterologous to the RNEA molecule. It follows that the absolute number of necessary events can be reduced since the probability to get suitable expression is higher.

The term “integration into the genome” is to be understood as stable integration of a nucleic acid molecule into the genome of an organism so that the nucleic acid molecule may be inherited over various subsequent generations of the respective organism.

The term “population” is to be understood as a plurality of organisms, preferably plants, preferably of the same genus, that are grown under similar, preferably identical conditions. The individual organisms of the population may be grown at various time points, preferably at the same time.

The term “independent primary transformant” is to be understood as organisms, preferably plants regenerated in one or more transformation experiments derived from not identical transformation events. Hence, from each transformation event only one plant is regenerated. The organisms in a population of independent transformant plants are therefore no genotypic clones but are derived each from distinct transformation events.

Populations of plant derived from the primary transformant plants of 2^{nd}, 3^{rd} or any other generation derived from plants or populations produced according to a method of the invention are also exhibiting a reduced coefficient of variation and/or reduced number of low or non expressing plants. Hence, the methods of the invention may also be used for reducing the number of low- or non-expressing plants in subsequent generations therefore lowering the number of low-expressing or silenced plants in a population in the n+1 generation.

A population of primary transformant plants exhibiting a reduced coefficient of variation of expression of the nucleic acid molecule of interest which is functionally linked to the RENA molecule compared to a population of primary transformant plants comprising a comparable, preferably a recombinant expression construct which is identical despite that it is not comprising a recombinant RENA molecule is of advantage as the number of non expressing or low expressing plants in the population exhibiting a reduced coefficient of variation is reduced. The number of plants that need to be regenerated in a transformation process in order to isolate at least one plant exhibiting the desired expression level may thereby be reduced. Plant transformation and regeneration is a very laborious process especially if plant species or germplasms are concerned that are recalcitrant to transformation and/or regeneration. Hence a transformation process that requires reduced number of regeneration of T1 plants is highly valuable.

A person skilled in the art is aware of methods for rendering a unidirectional to a bidirectional promoter and of methods to use the complement or reverse complement of a promoter sequence for creating a promoter having the same promoter specificity as the original sequence. Such methods are for example described for constitutive as well as inducible promoters by Xie et al. (2001) “Bidirectionalization of polar promoters in plants” nature biotechnology 19, pages 677-679. The authors describe that it is sufficient to add a minimal promoter to the 5′ end of any given promoter to receive a promoter controlling expression in both directions with same properties such as promoter specificity and strength. Hence a promoter functionally linked to a RENA as described above is functional in complement or reverse complement and therefore the RENA is functional in complement or reverse complement too.

In principal the RENA may be functionally linked to any promoter such as tissue specific, inducible, developmental specific or constitutive promoters. The respective RENA will lead to an enhanced reliability of expression of the heterologous nucleic acid under the control of the respective promoter to which the at least one RENA is functionally linked to.

Preferably, the one or more RENA is functionally linked to any heterologous promoter and will enhance reliability of expression of the nucleic acid molecule under control of said promoter. Constitutive promoters to be used in any method of the invention may be derived from plants, for example monocotyledonous or dicotyledonous plants, from bacteria and/or viruses or may be synthetic promoters. Constitutive promoters to be used are for example the PcUbi-Promoter from P. crispum (WO 2003102198), the ZmUbi-Promoter from Zea maize, AtNit-promoter from the A. thaliana gene At3g44310 encoding nitrilase 1, the 34S-promoter from figwort mosaic virus, the 35S-promoter from tobacco mosaic virus, the nos and ocs-promoter derived from Agrobacteria, the ScBV-promoter (U.S. Pat. No. 5,994,123), the SUPER-promoter (Lee et al. 2007, Plant. Phys.), the AtFNR-promoter from the A. thaliana gene At5g66190 encoding the ferredoxin NADH reductase, the ptxA promoter from Pisum sativum (WO2005085450), the AtTPT-promoter from the A. thaliana gene At5g46110 encoding the triose phosphate translocator, the bidirectional AtOASTL-promoter from the A. thaliana genes At4g14880 and At4g14890, the PRO0194 promoter from the A. thaliana gene At1g13440 encoding the glyceraldehyde-3-phosphate dehydrogenase, the PRO0162 promoter from the A. thaliana gene At3g52930 encoding the fructose-bis-phosphate aldolase, the AHAS-promoter (WO2008124495) or the CaffeoylCoA-MT promoter and the OsCP12 from rice (WO2006084868).

The promoter of the invention functionally linked to a heterologous RENA may be employed in any plant comprising for example moss, fern, gymnosperm or angiosperm, for example monocotyledonous or dicotyledonous plant. In a preferred embodiment said promoter of the invention functionally linked to a RENA may be employed in monocotyledonous or dicotyledonous plants, preferably crop plant such as corn, soy, canola, cotton, potato, sugar beet, rice, wheat, sorghum, barley, musa, sugarcane, miscanthus and the like. In a preferred embodiment of the invention, said promoter which is functionally linked to a RENA may be employed in monocotyledonous crop plants such as corn, rice, wheat, sorghum, musa, miscanthus, sugarcane or barley. In an especially preferred embodiment the promoter functionally linked to a RENA may be employed in dicotyledonous crop plants such as soy, canola, cotton, sugar beet or potato.

Methods for quantifying expression are known in the art. For example, the promoter functionally linked to at least one RENA molecule may be functionally linked to a marker gene such as GUS, GFP or luciferase and the activity of the respective protein encoded by the respective marker gene may be determined in the plant or part thereof. As a representative example, the method for detecting luciferase is described in detail below. Other methods are for example measuring the steady state level or synthesis rate of RNA of the nucleic acid molecule controlled by the promoter by methods known in the art, for example Northern blot analysis, qPCR, run-on assays or other methods described in the art.

A skilled person is aware of various methods for functionally linking two or more nucleic acid molecules. Such methods may encompass restriction/ligation, ligase independent cloning, recombineering, recombination or synthesis. Other methods may be employed to functionally link two or more nucleic acid molecules.

The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. For example, a RENA of the invention is in its natural environment functionally linked to its native promoter, whereas in the present invention it is linked to another promoter which might be derived from the same organism, a different organism or might be a synthetic promoter such as the SUPER-promoter. It may also mean that the RENA of the present invention is linked to its native promoter but the nucleic acid molecule under control of said promoter is heterologous to the promoter comprising its native RENA. It is in addition to be understood that the promoter and/or the nucleic acid molecule under the control of said promoter functionally linked to a RENA of the invention are heterologous to said RENA as their sequence has been manipulated by for example mutation such as insertions, deletions and the forth so that the natural sequence of the promoter and/or the nucleic acid molecule under control of said promoter is modified and therefore have become heterologous to a RENA of the invention. It may also be understood that the RENA is heterologous to the nucleic acid to which it is functionally linked when the RENA is functionally linked to its native promoter wherein the position of the RENA in relation to said promoter is changed so that the promoter exhibits enhanced reliability of expression after such manipulation.

A further embodiment of the invention is a method for producing a population of transgenic plants, preferably independent plants for example independent primary transformant plants with reduced percentage of low-expressing plants comprising the steps of
I) providing a recombinant nucleic acid construct comprising at least one RENA molecule as defined in above in i) to v) functionally linked to a promoter and/or a nucleic acid molecule of interest wherein at least the promoter is heterologous to the RENA molecule and
II) integrating said recombinant nucleic acid construct in the genome of plant cells, plants or parts thereof and
III) regenerating a population of independent primary transformant plants from the transgenic plant cells, plants or parts thereof produced in step II),

wherein the population of independent primary transformant plants has an at least 5% or 10%, preferably at least 20%, 25% or 30%, preferably at least 40% or 50%, more preferably at least 60%, 70% or 80%, even more preferably at least 90%, 95% or 100% reduced percentage of plants exhibiting low or non expression of the nucleic acid of interest compared to a population of plants, preferably independent primary transformant plants comprising in their genome a respective recombinant nucleic acid construct not comprising the respective RENA molecule as defined in i) to v).

Preferably the RENA molecule is heterologous to both the promoter and the nucleic acid of interest to which it is functionally linked.

The percentage of non- or low-expressers is calculated relative to the mean expression level of the nucleic acid of interest derived from the respective construct in a population of plants. In a preferred embodiment, the percentage of non- or low-expressers in a population is defined as an at least 5% or 10%, preferably at least 20%, 25% or 30%, preferably at least 40% or 50%, more preferably at least 60%, 70% or 80%, even more preferably at least 90%, 95% or 100% reduction of plants expressing lower than 5%, preferably lower than 10%, more preferably lower than 20% or 25%, even more preferably 30%, most preferably 40% or 50% of the mean.

A method for reducing the number of low expressing plants, preferably independent primary transformant plants, comprising the steps of
I) providing a recombinant nucleic acid construct comprising at least one RENA molecule as defined above under i) to v) functionally linked to a promoter and/or a nucleic acid molecule of interest wherein at least the promoter is heterologous to the RENA molecule and
II) integrating said recombinant nucleic acid construct in the genome of plant cells, plants or parts thereof and
III) regenerating independent primary transformant plants from the transgenic plant cells, plants or parts thereof produced in step II),

wherein the number of low expressing independent primary transformant plants is reduced by at least 5% or 10%, preferably at least 20%, 25% or 30%, preferably at least 40% or 50%, more preferably at least 60%, 70% or 80%, even more preferably at least 90%, 95% or 100% compared to a population of independent primary transformant plants comprising in their genome a respective recombinant nucleic acid not comprising the respective RENA molecule as defined above under i) to v) is a further embodiment of the invention.

The methods as described above, wherein the RENA molecule is comprised in an expression construct or vector functionally linked to a promoter and/or a nucleic acid molecule of interest wherein at least the promoter is heterologous to the at least one RENA molecule, preferably the RENA molecule is heterologous to both the promoter and the nucleic acid of interest is an additional embodiment of the invention.

The methods as described above wherein said at least one RENA is located 2500 bp or less, preferentially 2000 bp or less, more preferred 1500 bp or less, even more preferred 1000 bp or less and most preferred 500 bp or less away from the transcription start site of said heterologous nucleic acid molecule of interest to which it is functionally linked is also one embodiment of the invention. Preferably the at least one RENA is located upstream of the translational start site of the nucleic acid molecule to which it is functionally linked. In another embodiment of the invention the at least one RENA is located within a 5′UTR of the nucleic acid molecule of interest to which it is functionally linked. The 5′UTR may be homologous or heterologous to the promoter and/or nucleic acid of interest of the construct used in the methods of the invention.

A further embodiment of the invention is a recombinant expression construct comprising at least one RENA molecule as defined above under points i) to vi) functionally linked to a promoter and/or a nucleic acid molecule of interest wherein at least the promoter is heterologous to the RENA molecule. In one embodiment of the invention the RENA is heterologous to both the promoter and the nucleic acid of interest comprised in the construct of the invention.

A further embodiment of the invention is a method as described above or the recombinant expression construct as described above wherein the promoter is a constitutive promoter, a tissue-specific or tissue-preferential promoter, a developmental-specific or developmental-preferential promoter or an inducible promoter. A recombinant expression vector comprising one or more recombinant expression constructs as described above is also an embodiment of the invention.

A further embodiment of the invention is a transgenic cell or transgenic plant or part thereof, for example propagation material comprising
a) at least one RENA molecule as defined above under i) to v) which is functionally linked to a heterologous promoter and/or a heterologous nucleic acid of interest to be expressed or
b) a recombinant expression construct as described above or
c) a recombinant expression vector as described above.

The transgenic cell or transgenic plant or part thereof may be selected from the group consisting of bacteria, fungi, yeasts or plants.

Transgenic parts or propagation material as meant herein comprise all tissues and organs, for example leaf, stem and fruit as well as material that is useful for propagation and/or regeneration of plants such as cuttings, scions, layers, branches or shoots comprising the respective RENA functionally linked to a heterologous promoter and/or nucleic acid of interest, recombinant expression construct or recombinant vector.

A use of a RENA molecule as defined above under point i) to v) or the recombinant expression construct or recombinant expression vector as defined in above for reducing the coefficient of variation in a population of primary transformant plants or reducing the percentage of low-expressing plants in a population of primary transformant plants is a further embodiment of the invention.

A method for the production of an agricultural product by introducing a RENA molecule as defined above under point i) to v) or the recombinant construct or recombinant vector as defined above into a plant, growing the plant, harvesting and processing the plant or parts thereof is a further embodiment of the invention.

The method of the invention may be applied to any plant, for example gymnosperm or angiosperm, preferably angiosperm, for example dicotyledonous or monocotyledonous plants, preferably dicotyledonous plants. Preferred monocotyledonous plants are for example corn, wheat, rice, barley, sorghum, musa, sugarcane, miscanthus and brachypodium, especially preferred monocotyledonous plants are corn, wheat and rice. Preferred dicotyledonous plants are for example soy, rape seed, canola, linseed, cotton, potato, sugar beet, tagetes and Arabdopsis, especially preferred dicotyledonous plants are soy, rape seed, canola and potato.

The one or more RENA molecule may be introduced into the plant or part thereof by means of particle bombardment, protoplast electroporation, virus infection, Agrobacterium mediated transformation or any other approach known in the art. The RENA molecule may be introduced integrated for example into a plasmid or viral DNA or viral RNA. The RENA molecule may also be comprised on a BAC, YAC or artificial chromosome prior to introduction into the plant or part of the plant. It may be also introduced as a linear nucleic acid molecule comprising the RENA sequence wherein additional sequences may be present adjacent to the RENA sequence on the nucleic acid molecule. These sequences neighboring the RENA sequence may be from about 20 bp, for example 20 bp to several hundred base pairs, for example 100 bp or more and may facilitate integration into the genome for example by homologous recombination. Any other method for genome integration may be employed, such as targeted integration approaches, such as homologous recombination or random integration approaches, such as illegitimate recombination.

The endogenous expressed nucleic acid to which the RENA molecule may be functionally linked may be any nucleic acid, preferably any constitutively expressed nucleic acid molecule. The nucleic acid molecule may be a protein coding nucleic acid molecule or a non coding molecule such as antisense RNA, rRNA, tRNA, miRNA, ta-siRNA, siRNA, dsRNA, snRNA, snoRNA or any other noncoding RNA known in the art.

In another preferred embodiment said one or more RENA is functionally linked to a promoter close to the transcription start site of said heterologous nucleic acid molecule.

Close to the transcription start site as meant herein comprises functionally linking one or more RENA to a promoter 2500 bp or less, preferentially 2000 bp or less, more preferred 1500 bp or less, even more preferred 1000 bp or less and most preferred 500 bp or less away from the transcription start site of said heterologous nucleic acid molecule. It is to be understood that the RENA may be integrated upstream or downstream in the respective distance from the transcription start site of the respective promoter. Hence, the one or more RENA must not necessarily be included in the transcript of the respective heterologous nucleic acid under control of the promoter the one or more RENA is functionally linked to. Preferentially the one or more RENA is integrated downstream of the transcription start site of the respective promoter. The integration site downstream of the transcription start site may be in the 5′ UTR, the 3′ UTR, an exon or intron or it may replace an intron or partially or completely the 5′ UTR or 3′ UTR of the heterologous nucleic acid under the control of the promoter. Preferentially the one or more RENA is integrated in the 5′ UTR or an intron or the RENA is replacing an intron or a part or the complete 5′UTR, most preferentially it is integrated in the 5′UTR of the respective heterologous nucleic acid.

A further embodiment of the invention is the use of the RENA as defined above in i) to v) or the recombinant construct or recombinant vector as defined above for enhancing reliability of expression in plants or parts thereof.

In a preferred embodiment, the RENAs having SEQ ID 1 to 16, 93 to 4243, 16160 to 35773, 38106 to 110789 and their functional homologs as defined above under i) to v) are used in methods for reducing the coefficient of variation in a population of primary transformant dicotyledonous plants or methods for producing a population of primary transformant dicotyledonous plants with reduced percentage of low-expressing plants, the RENAs having SEQ ID 4244 to 16159, 35774 to 38105 and 11160 to 113579 and their functional homologs as defined above under i) to v) are used in methods for reducing the coefficient of variation in a population of primary transformant monocotyledonous plants or methods for producing a population of primary transformant monocotyledonous plants with reduced percentage of low-expressing plants. In an especially preferred embodiment, the RENAs are used in the plant family or plant genus, they are derived from: Arabidopsis thaliana: SEQ ID NO 1 to 15 and 94 to 4243, Petrosilenum crispum: SEQ ID NO 16, Zea mays: SEQ ID NO 4244 to 9134, Oryza sativa: SEQ ID NO 9135 to 13754, Brachypodium distachyon: SEQ ID NO 13755 to 16159, Glycine max: SEQ ID NO 16160 to 27745, Medicago truncatula: SEQ ID NO 27746 to 35773, Sorghum bicolor: SEQ ID NO 35774 to 38105, Arabidopsis lyrata: SEQ ID NO 38106 to 42162, Manihot esculentum: SEQ ID NO 42163 to 48459, Ricinus communis: SEQ ID NO 48460 to 52747, Populus trichocarpa: SEQ ID NO 52748 to 61982, Cucumis sativus: SEQ ID NO 61983 to 68543, Prunus persica: SEQ ID NO 68544 to 73752, Carica papaya: SEQ ID NO 73753 to 77766, Citrus sinensis: SEQ ID NO 77767 to 83845, Citrus clementina: SEQ ID NO 83846 to 89915, Eucalyptos grandes: SEQ ID NO 89916 to 95632, Vitis vinifera: SEQ ID NO 95633 to 100567, Mimulus guttatus: SEQ ID NO 100568 to 106070, Aquilegia coerula: SEQ ID NO 106071 to 110789, Setaria italica: SEQ ID NO 110790 to 113579, Selaginella moellendorfii: SEQ ID NO 113580 to 113761, Physcomitrella patens: SEQ ID NO 113762 to 116585 and Volvox carteri: 116586 to 116666.

### DEFINITIONS

Abbreviations: RENA—nucleic acid expression enhancing nucleic acid, GFP—green fluorescence protein, GUS—beta-Glucuronidase, BAP—6-benzylaminopurine; 2,4-D-2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3—Gibberellic acid; Timentin™: ticarcillin disodium/clavulanate potassium, microl: Microliter.

It is to be understood that this invention is not limited to the particular methodology or protocols. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed plant cell.

Enhanced expression: “enhance” or “increase” the expression of a nucleic acid molecule in a plant cell are used equivalently herein and mean that the level of expression of a nucleic acid molecule in a plant, part of a plant or plant cell is higher compared to a reference plant, part of the plant or plant cell. The terms “enhanced” or “increased” as used herein are synonymous and mean herein higher, preferably significantly higher expression of the nucleic acid molecule to be expressed. As used herein, an “enhancement” or “increase” of the level of an agent such as a protein, mRNA or RNA means that the level is increased relative to a substantially identical plant, part of a plant or plant cell grown under substantially identical conditions. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a cell or organism lacking a recombinant nucleic acid molecule of the invention. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254). As one example for quantifying the activity of a protein, the detection of luciferase activity is described in the Examples below.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is —optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator or a RENA) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as used herein includes “any process by which a strand of nucleic acid molecule joins with a complementary strand through base pairing.” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. Stringent conditions, are known to those skilled in the art and can be found in Current Protocols in Molecular Biology, John Wiley & Sons, N.Y. (1989), 6.3.1-6.3.6.

“Identity”: “Identity” when used in respect to the comparison of two or more nucleic acid or amino acid molecules means that the sequences of said molecules share a certain degree of sequence similarity, the sequences being partially identical.

To determine the percentage identity (homology is herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid).

The amino acid residues or nucleic acid molecules at the corresponding amino acid positions or nucleotide positions are then compared. If a position in one sequence is occupied by the same amino acid residue or the same nucleic acid molecule as the corresponding position in the other sequence, the molecules are homologous at this position (i.e. amino acid or nucleic acid “homology”as used in the present context corresponds to amino acid or nucleic acid “identity”. The percentage identity between the two sequences is a function of the number of identical positions shared by the sequences (i.e. % homology=number of identical positions/total number of positions ×100). The terms “homology” and “identity” are thus to be considered as synonyms.

For the determination of the percentage identity of two or more amino acids or of two or more nucleotide sequences several computer software programs have been developed. The identity of two or more sequences can be calculated with for example the software fasta, which presently has been used in the version fasta 3 (W. R. Pearson and D. J. Lipman, PNAS 85, 2444 (1988); W. R. Pearson, Methods in Enzymology 183, 63 (1990); W. R. Pearson and D. J. Lipman, PNAS 85, 2444 (1988); W. R. Pearson, Enzymology 183, 63 (1990)). Another useful program for the calculation of identities of different sequences is the standard blast program, which is included in the Biomax pedant software (Biomax, Munich, Federal Republic of Germany). This leads unfortunately sometimes to suboptimal results since blast does not always include complete sequences of the subject and the query. Nevertheless as this program is very efficient it can be used for the comparison of a huge number of sequences. The following settings are typically used for such a comparisons of sequences:

-p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; —W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence identity are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which is said to have 80% identity with sequence SEQ ID NO: 1 at the nucleic acid level is understood as meaning a sequence which, upon comparison with the sequence represented by SEQ ID NO: 1 by the above program “Needle” with the above parameter set, has a 80% identity. Preferably the identity is calculated on the complete length of the query sequence, for example SEQ ID NO:1.

Intron: refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are —beside other sequences—essential for the intron to be accurately spliced. These sequences are the 5′ splice-site, the 3′ splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron). The functional elements of an intron is comprising sequences that are recognized and bound by the specific protein components of the spliceosome (e.g. splicing consensus sequences at the ends of introns). The interaction of the functional elements with the spliceosome results in the removal of the intron sequence from the premature mRNA and the rejoining of the exon sequences. Introns have three short sequences that are essential—although not sufficient—for the intron to be accurately spliced. These sequences are the 5′ splice site, the 3′ splice site and the branch point. The branchpoint sequence is important in splicing and splice-site selection in plants. The branchpoint sequence is usually located 10-60 nucleotides upstream of the 3′ splice site.

Isogenic: organisms (e.g., plants), which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

RENA: see “Reliability enhancing nucleic acid”.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Reliability enhancing nucleic acid (RENA): The term “reliability enhancing nucleic acid” refers to a sequence and/or a nucleic acid molecule of a specific sequence having the intrinsic property to enhance reliability of expression of a nucleic acid of interest under the control of a promoter to which the RENA is functionally linked. Unlike promoter sequences, the RENA as such is not able to drive expression. In order to fulfill the function of enhancing reliability of expression of a nucleic acid molecule functionally linked to the RENA, the RENA itself has to be functionally linked to a promoter. In distinction to enhancer sequences known in the art, the RENA is acting in cis but not in trans and has to be located close to the transcription start site of the nucleic acid to be expressed.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end.

It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: is generally understood as meaning any eukaryotic single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. Such promoters can for example be found in the following public databases http://www.grassius.org/grasspromdb.html, http://mendel.cs.rhul.ac.uk/mendel.php?topic=plantprom, http://ppdb.gene.nagoya-u.ac.jp/cgi-Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

Promoter specificity: The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which were isolated from their natural environment, such as their genomic localization in a wild type plant or the nucleic acids they are functionally linked in a wild type plant. The term also comprises nucleic acid molecules which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In a especially preferred embodiment the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism that said organism is not changed, mutated, or otherwise manipulated by man. With respect to a polypeptide or nucleic acid sequence, that the polypeptide or nucleic acid sequence is naturally occurring or available in at least one naturally occurring organism which is not changed, mutated, or otherwise manipulated by man.

### EXAMPLES

### Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, Calif., USA) using the Sanger technology (Sanger et al., 1977). Unless described otherwise, chemicals and reagents were obtained from Sigma Aldrich (Sigma Aldrich, St. Louis, USA), from Promega (Madison, Wis., USA), Duchefa (Haarlem, The Netherlands) or Invitrogen (Carlsbad, Calif., USA). Restriction endonucleases were from New England Biolabs (Ipswich, Mass., USA) or Roche Diagnostics GmbH (Penzberg, Germany). Oligonucleotides were synthesized by Eurofins MWG Operon (Ebersberg, Germany).

### Example 1

### Identification of Reliability Enhancing Nucleic Acids (RENAs)

1.1 Identification of RENA Molecules from A. thaliana Genes

Using publicly available genomic DNA sequences (e.g. http://www.ncbi.nlm.nih.gov/genomes/PLANTS/PlantList.html) and transcript expression data (e.g. http://www.weigelworld.org/resources/microarray/AtGenExpress/), a set of 16 potential RENA candidates deriving from Arabidopsis thaliana transcripts were selected for detailed analyses. In addition, a putative RENA molecule deriving from parsley was also included in the analysis. The candidates were named as follows:

TABLE 1
Reliability Enhancing Nucleic Acid (RENA) candidates

| RENA | | | SEQ |
|---|---|---|---|
| name | Locus | Annotation | ID NO |
| RENA1 | At1g52370 | ribosomal protein L22 family protein | 1 |
| RENA2 | At1g03280 | transcription initiation factor IIE | 2 |
| | | (TFIIE) alpha subunit family protein | |
| RENA3 | At5g06960 | classic OBF5 (OCS-ELEMENT | 3 |
| | | BINDING FACTOR 5) | |
| RENA4 | At1g54290 | eukaryotic translation initiation factor | 4 |
| | | SUI1, putative | |
| RENA5 | At2g47170 | ADP-ribosylation factor 1 (ARF1) | 5 |
| RENA6 | At1g56070 | elongation factor 2, putative/EF-2, | 6 |
| | | putative | |
| RENA7 | At5g54760 | eukaryotic translation initiation factor | 7 |
| | | SUI1, putative | |
| RENA8 | At4g02890 | polyubiquitin (UBQ14) | 8 |
| RENA9 | At1g62290 | aspartyl protease family protein | 9 |
| RENA10 | At1g65090 | expressed protein | 10 |
| RENA11 | At2g27040 | PAZ domain-containing protein | 11 |
| RENA12 | At1g01170 | ozone-responsive stress-related | 12 |
| | | protein, putative | |
| RENA13 | At5g63190 | MA3 domain-containing protein | 13 |
| RENA14 | At5g07830 | glycosyl hydrolase family 79 | 14 |
| | | N-terminal domain-containing | |
| | | protein similar to | |
| | | beta-glucuronidase AtGUS2 | |
| RENA15 | At2g04520 | eukaryotic translation initiation factor | 15 |
| | | 1A, putative/elF-1A | |
| RENA16 | | Petroselinum crispum gene Pcubi4-2 | 16 |
| | | for polyubiquitin | |

1.2 Isolation of the RENA Candidates

Genomic DNA was extracted from A. thaliana green tissue using the Qiagen DNeasy Plant Mini Kit (Qiagen, Hilden, Germany). For the putative RENA molecule with the SEQ ID NO16, DNA of the vector construct 1bxPcUbi4-2GUS (WO 2003102198) was used. Genomic DNA fragments containing putative RENA molecules were isolated by conventional polymerase chain reaction (PCR). The polymerase chain reaction comprised 16 sets of primers (Table 2). Primers were designed on the basis of the A. thaliana genome sequence with a multitude of RENA candidates. The nucleotide sequence of the vector construct 1bxPcUbi4-2GUS (WO 2003102198) was used for the design of primers (SEQ ID NO47 and NO48) for amplification of the RENA candidate with SEQ ID NO16 (Table 2). The polymerase chain reaction followed the protocol outlined by Phusion High Fidelity DNA Polymerase (Cat No F-540L, New England Biolabs, Ipswich, Mass., USA). The isolated DNA was used as template DNA in a PCR amplification using the following primers:

TABLE 2
Primer sequences

| | | | PCR |
|---|---|---|---|
| | | | yielding |
| | | SEQ ID | SEQ ID |
| Primer name | Sequence | NO | NO |
| RENA1_for | TATAGGTACCCGGGCATTTTCGTCTAAGC | 17 | 1 |
| RENA1_rev | TATACCATGGACCCTAAACCTTAGTCTATGCTTCT | 18 | |
| RENA2_for | TATAGGTACCTCTCTCCCCCTCTCTTGAACTT | 19 | 2 |
| RENA2_rev | TATACCATGGGCAGTATTAGGTTCGTGTTTGATAC | 20 | |
| RENA3_for | TATAGGTACCGTTAAGCTCAAAGAATCCGTTCT | 21 | 3 |
| RENA3_rev | TATACCATGGATCGTGGTACCCTAGATGGAGTA | 22 | |
| RENA4_for | TATAGGTACCCTCGAAACCCTAATCTCTTCTTG | 23 | 4 |
| RENA4_rev | TATACCATGGTACAAGGGACAGTAAATCGACAAG | 24 | |
| RENA5_for | tttttggtaccttagatctcgtgccgtcgtgcga | 25 | 5 |
| RENA5_rev | tttttccatggtttgatcaagcctgttcaca | 26 | |
| RENA6_for | tttttggtacctttctctcgttctcatctttctctct | 27 | 6 |
| RENA6_rev | taatagatatctttgtcaaacttttgattgtcacct | 28 | |
| RENA7_for | aaaaaggtacctcatcagatcttcaaaaccccaa | 29 | 7 |
| RENA7_rev | aaaaaccatggtgatttgagggtagtactaaccgggaa | 30 | |
| RENA8_for | tatatggtaccaaatcgttctttcaaatctctca | 31 | 8 |
| RENA8_rev | ttataccatggtctgtaattcacaaaaaactgaga | 32 | |
| RENA9_for | aataatggtacctggtgcttaaacactctggtgagt | 33 | 9 |
| RENA9_rev | aataatccatggtttgacctacaaaatcaaagcagtca | 34 | |
| RENA10_for | ttttttggtaccagttctttgctttcgaagttgc | 35 | 10 |
| RENA10_rev | ttttttccatggtactacgtactgttttcaattct | 36 | |
| RENA11_for | tatataggtaccagggtttcgtttttgtttca | 37 | 11 |
| RENA11_rev | tatataccatggttatctcctgctcaaagaaacca | 38 | |
| RENA12_for | tatataggtaccactgtttaagcttcactgtct | 39 | 12 |
| RENA12_rev | tatataccatggtttcttctaaagctgaaagt | 40 | |
| RENA13_for | ttttttggtacctttcacgatttggaatttga | 41 | 13 |
| RENA13_rev | ttttttccatggtctacaacattaaaacgaccatta | 42 | |
| RENA14_for | aataaaggtaccgtccagaattttctccattga | 43 | 14 |
| RENA14_rev | aataaaccatggtcttcactatccaaagctctca | 44 | |
| RENA15_for | ttttatggtacctagcttaatctcagattcgaatcgt | 45 | 15 |
| RENA15_rev | ttttatccatggtagtatctacataccaatcatacaaatg | 46 | |
| RENA16_for | tttatggtaccagccgcaagactcctttcagattct | 47 | 16 |
| RENA16_rev | aaattccatggtagctgtcaaaacaaaaacaaaaatcga | 48 | |

Amplification During the PCR was Carried Out with the Following Composition (50 Micro):

3.00 microl A. thaliana genomic DNA (50 ng/microl genomic DNA, 5 ng/microl vector construct) 10.00 microl 5× Phusion HF Buffer

4.00 microl dNTP (2.5 mM)

2.50 microl for Primer (10 microM)

2.50 microl rev Primer (10 microM)

0.50 microl Phusion HF DNA Polymerase (2 U/microl)

A touch-down approach was employed for the PCR with the following parameters: 98.0° C. for 30 sec (1 cycle), 98.0° C. for 30 sec, 56.0° C. for 30 sec and 72.0° C. for 60 sec (4 cycles), 4 additional cycles each for 54.0° C., 51.0° C. and 49.0° C. annealing temperature, followed by 20 cycles with 98.0° C. for 30 sec, 46.0° C. for 30 sec and 72.0° C. for 60 sec (4 cycles) and 72.0° C. for 5 min. The amplification products was loaded on a 2% (w/v) agarose gel and separated at 80V. The PCR products were excised from the gel and purified with the Qiagen Gel Extraction Kit (Qiagen, Hilden, Germany). Following a DNA restriction digest with KpnI (10 U/microl) and NcoI (10 U/microl) or EcoRV (10 U/microl) restriction endonuclease, the digested products were again purified with the Qiagen Gel Extraction Kit (Qiagen, Hilden, Germany).

1.3 Vector Construction with Potential RENA Molecules

Using the Multisite Gateway System (Invitrogen, Carlsbad, Calif., USA), the promoter::RENA::reporter-gene cassettes were assembled into binary constructs for plant transformation. In one set of reporter gene constructs the A. thaliana p-AtNit1 promoter (At3g44310, GenBank X86454; WO03008596, with the prefix p- denoting promoter) was used, in the other set of reporter gene constructs the A. thaliana p-AtPXR (At1g48130, GenBank AC023673.3; WO2006089950; with the prefix p- denoting promoter) seed specific promoter was used. Firefly luciferase (Promega, Madison, Wis., USA) was utilized as reporter protein for quantitatively determining the expression enhancing effects of the putative RENA molecules to be analyzed. The pENTR/A vector holding the p-AtNit1 promoter was cloned via site specific recombination (BP-reaction) between the pDONR/A vector and p-AtNit1 amplification products with primers p-AtNit1-for and p-AtNit1-rev (Table 3) on genomic DNA (see above) with site specific recombination sites at either end according to the manufacturers manual (Invitrogen, Carlsbad, Calif., USA). Positive pENTR/A clones underwent sequence analysis to ensure correctness of the p-AtNit1 promoter.

The pENTR/A vector holding the p-AtPXR promoter was cloned via site specific recombination (BP-reaction) between the pDONR/A vector and p-AtPXR amplification products with primers p-AtPXR-for and p-AtPXR-rev (Table 3) on genomic DNA (see above) with site specific recombination sites at either end according to the manufacturers manual (Invitrogen, Carlsbad, Calif., USA). Positive pENTR/A clones underwent sequence analysis to ensure correctness of p-AtPXR promoter.

TABLE 3
Primer sequences (p-AtNit1 and p-AtPXR)

| Primer name | Sequence | SEQ ID NO. |
|---|---|---|
| p-AtNit1-for | ggggacaactttgtatagaaaagttg | 73 |
| | tcgagaccagatgttttacacttga | |
| p-AtNit1-rev | ggggactgcttttttgtacaaacttg | 74 |
| | gacactcagagacttgagagaagca | |
| p-AtPXR-for | ggggacaactttgtatagaaaagttg | 75 |
| | gccacatcatgtttagacttatc | |
| p-AtPXR-rev | ggggactgcttttttgtacaaacttg | 76 |
| | tttaccttttatatttatatatag | |

An ENTR/B vector containing the firefly luciferase coding sequence (Promega, Madison, Wis., USA) followed by the t-nos nopaline synthase transcriptional terminator (Genbank V00087) was generated. RENA candidate PCR fragments (see above) were cloned separately upstream of the firefly luciferase coding sequence using KpnI and NcoI or EcoRV restriction enzymes. The resulting pENTR/B vectors are summarized in table 4, with promoter molecules having the prefix p-, coding sequences having the prefix c-, and terminator molecules having the prefix t-.

TABLE 4
All pENTR/B vectors plus and minus RENA candidates

| pENTR/B | Composition of the partial expression cassette |
|---|---|
| vector | SEQ ID NO::reporter gene::terminator |
| LJK1 | MCS::c-LUC::t-nos |
| LJH45 | SEQ ID NO1::c-LUC::t-nos |
| LJH46 | SEQ ID NO2::c-LUC::t-nos |
| LJH47 | SEQ ID NO3::c-LUC::t-nos |
| LJH48 | SEQ ID NO4::c-LUC::t-nos |
| LJK57 | SEQ ID NO5::c-LUC::t-nos |
| LJK44 | SEQ ID NO6::c-LUC::t-nos |
| LJK58 | SEQ ID NO7::c-LUC::t-nos |
| LJK47 | SEQ ID NO8::c-LUC::t-nos |
| LJK19 | SEQ ID NO9::c-LUC::t-nos |
| LJK20 | SEQ ID NO10::c-LUC::t-nos |
| LJK33 | SEQ ID NO11::c-LUC::t-nos |
| LJK36 | SEQ ID NO12::c-LUC::t-nos |
| LJK32 | SEQ ID NO13::c-LUC::t-nos |
| LJK22 | SEQ ID NO14::c-LUC::t-nos |
| LJK31 | SEQ ID NO15::c-LUC::t-nos |
| LJK4 | SEQ ID NO16::c-LUC::t-nos |

The pENTR/C vector was constructed by introduction of a gene expression cassette carrying a mutated AHAS gene driven by the parsley ubiquitin promoter PcUbi4-2, mediating tolerance to imidazolinone herbicides for detecting transgenic plant lines. By performing a site specific recombination (LR-reaction), the pENTR/A, pENTR/B and the pENTR/C carrying the selectable marker cassette were combined with the pSUN destination vector according to the manufacturers (Invitrogen, Carlsbad, Calif., USA) Multisite Gateway manual.

The reactions yielded 1 binary vector with p-AtNit1 promoter, the firefly luciferase coding sequence c-LUC, the t-nos terminator and the selectable marker cassette, as well as 1 binary vector with p-AtPXR promoter, the firefly luciferase coding sequence c-LUC, the t-nos terminator and the selectable marker cassette.

In addition the reaction yielded 14 vectors harboring the p-AtNit1 promoter together with SEQ ID NO1, NO2, NO3, NO4, NO5, NO6, NO7, NO8, NO9, NO10, NO11, NO12, NO13 or NO16 immediately upstream of the firefly luciferase coding sequence (Table 5), for which the combination with SEQ ID NO1 is given exemplary (SEQ ID NO79). Except for varying SEQ ID NO2 to NO13 and NO16, the nucleotide sequence is identical in vectors LJH60-63, LJK139, LJK141-144 and LJK311-315.

In addition the reaction yielded 7 vectors harboring the p-AtPXR promoter together with SEQ ID NO9, NO10, NO11, NO12, NO13, NO14 or NO15 immediately upstream of the firefly luciferase coding sequence (Table 5), for which the combination with SEQ ID NO9 is given exemplary (SEQ ID NO80). Except for varying SEQ ID NO10 to NO15, the nucleotide sequence is identical in vectors LJK156-162.

The resulting plant transformation vectors are summarized in table 5:

TABLE 5
Plant expression vectors for A. thaliana transformation

| plant | | |
|---|---|---|
| expression | Composition of the expression cassette | SEQ |
| vector | Promoter::SEQ ID NO::reporter gene::terminator | ID NO |
| LJK138 | p-AtNit1::-::c-LUC::t-nos | |
| LJH63 | p-AtNit1::SEQ ID NO1::c-LUC::t-nos | 79 |
| LJH60 | p-AtNit1::SEQ ID NO2::c-LUC::t-nos | |
| LJH61 | p-AtNit1::SEQ ID NO3::c-LUC::t-nos | |
| LJH62 | p-AtNit1::SEQ ID NO4::c-LUC::t-nos | |
| LJK143 | p-AtNit1::SEQ ID NO5::c-LUC::t-nos | |
| LJK141 | p-AtNit1::SEQ ID NO6::c-LUC::t-nos | |
| LJK144 | p-AtNit1::SEQ ID NO7::c-LUC::t-nos | |
| LJK142 | p-AtNit1::SEQ ID NO8::c-LUC::t-nos | |
| LJK139 | p-AtNit1::SEQ ID NO16::c-LUC::t-nos | |
| LJK148 | p-AtPXR::-::c-LUC::t-nos | |
| LJK156 | p-AtPXR::SEQ ID NO9::c-LUC::t-nos | 80 |
| LJK157 | p-AtPXR::SEQ ID NO10::c-LUC::t-nos | |
| LJK162 | p-AtPXR::SEQ ID NO11::c-LUC::t-nos | |
| LJK160 | p-AtPXR::SEQ ID NO12::c-LUC::t-nos | |
| LJK159 | p-AtPXR::SEQ ID NO13::c-LUC::t-nos | |
| LJK161 | p-AtPXR::SEQ ID NO14::c-LUC::t-nos | |
| LJK158 | p-AtPXR::SEQ ID NO15::c-LUC::t-nos | |
| LJK311 | p-AtNit::SEQ ID NO9::c-LUC::t-nos | |
| LJK312 | p-AtNit::SEQ ID NO10::c-LUC::t-nos | |
| LJK313 | p-AtNit::SEQ ID NO11::c-LUC::t-nos | |
| LJK314 | p-AtNit::SEQ ID NO12::c-LUC::t-nos | |
| LJK315 | p-AtNit::SEQ ID NO13::c-LUC::t-nos | |

All plant transformation vectors from table 5 were subsequently used to generate stable transgenic A. thaliana plants (see 2.1).

### Example 2

### Screening for RENA Molecules Enhancing Reliability of Gene Expression in Transgenic A. thaliana Plants

2.1 Generation of Stable Transgenic A. thaliana Plants

Expression constructs from table 5 in example 1 containing RENA candidate molecules were stably transformed into Arabidopsis thaliana plants along with RENA-less control constructs. In order to generate transgenic A. thaliana plants, Agrobacterium tumefaciens (strain C58C1 pGV2260) was transformed with the various vector constructs described above. For A. thaliana transformation, the Floral Dip method was employed (Clough and Bent, 1998, Plant Journal 16: 735-743). T1 transgenic plants were selected by germinating and growing seedlings under imidazolinone herbicide selection.

2.2 Plant Analysis

Leaf material of adult transgenic A. thaliana plants was sampled, frozen in liquid nitrogen and subjected to Luciferase reporter gene assays (amended protocol after Ow et al., 1986). After grinding the frozen tissue samples were resuspended in 800 microl of buffer I (0.1 M Phosphate buffer pH7.8, 1 mM DTT (Sigma Aldrich, St. Louis, Mo., USA), 0.05% Tween 20 (Sigma Aldrich, St. Louis, Mo., USA)) followed by centrifugation at 10 000 g for 10 min. 75 microl of the aqueous supernatant were transferred to 96-well plates. After addition of 25 microl of buffer II (80 mM gycine-glycyl (Carl Roth, Karlsruhe, Germany), 40 mM MgSO₄ (Duchefa, Haarlem, The Netherlands), 60 mM ATP (Sigma Aldrich, St. Louis, Mo., USA), pH 7.8) and D-Luciferin to a final concentration of 0.5 mM (Cat No: L-8220, BioSynth, Staad, Switzerland), luminescence was recorded in a MicroLumat Plus LB96V (Berthold Technologies, Bad Wildbad, Germany) yielding the unit relative light unit RLU per minute (RLU/min).

In order to normalize the luciferase activity between samples, the protein concentration was determined in the aqueous supernatant in parallel to the luciferase activity (adapted from Bradford, 1976, Anal. Biochem. 72, 248). 5 microl of the aqueous cell extract in buffer I were mixed with 250 microl of Bradford reagent (Sigma Aldrich, St. Louis, Mo., USA), incubated for 10 min at room temperature. Absorption was determined at 595 nm in a plate reader (Thermo Electron Corporation, Multiskan Ascent 354). The total protein amounts in the samples were calculated with a previously generated standard concentration curve. Values resulting from a ratio of RLU/min and mg protein/ml sample were averaged for transgenic plants harboring identical constructs and coefficient of variation was calculated to assess the impact of RENA molecule presence over RENA-less reporter gene constructs.

Constructs harboring RENAs with SEQ ID NO1-NO8 and NO16 under control of the p-AtNit1 promoter (LJH60-63, LJK139 and LJK141-144) show significantly higher reliability measured as a reduced coefficient of variation and a lower percentage of non- or low-expressers in the population of primary transformants than the RENA-less construct LJK138. Constructs harboring RENAs with SEQ ID NO9-NO15 under control of the p-AtPXR promoter (LJK156-LJK161) show significantly higher reliability measured as a reduced coefficient of variation and a lower percentage of non- or low-expressers in the population of primary transformants than the RENA-less construct LJK148.

Constructs harboring RENAs with SEQ ID NO9-NO13 under control of the p-AtNit1 promoter showed significantly higher reliability measured as a reduced coefficient of variation in leaves, flowers and siliques (FIG. 1). Table 6 shows an overview over the percentage of low-expressers calculated relative to the mean expression level of the respective construct in A. thaliana leaves.

TABLE 6
Percentage of low-expressers in a population (calculation
relative to mean expression value of the population);
A. thaliana leaves; p-AtNit1

| | Fraction of low expressers (<x% mean) | | | | | | |
|---|---|---|---|---|---|---|---|
| | 5% | 10% | 20% | 30% | 40% | 50% | 60% |
| | mean | mean | mean | mean | mean | mean | mean |
| LJK310 | 40 | 40 | 6.7 | 6.7 | 6.7 | 20 | 53.3 |
| LJK311 | 0 | 0 | 0 | 0 | 0 | 6.7 | 6.7 |
| LJK312 | 0 | 0 | 0 | 0 | 0 | 0 | 0 |
| LJK313 | 0 | 0 | 0 | 0 | 0 | 0 | 0 |
| LJK314 | 0 | 0 | 0 | 0 | 0 | 0 | 0 |
| LJK315 | 0 | 0 | 0 | 0 | 0 | 0 | 6.7 |

### Example 3

### Test of RENA Molecules for Enhanced Reliability of Gene Expression in Oilseed Rape Plants

This example illustrates that RENA molecules can be used across species to enhance reliability of gene expression in all tissues tested compared to a RENA-less promoter-only approach.

3.1 Selection of RENA Constructs for B. napus Transformation

RENA molecules with SEQ ID NO5, NO6, NO7, NO8 and NO16 and a RENA-less control under control of the p-AtNit1 promoter were selected for determining enhanced reliability of gene expression in transgenic oilseed rape leaves, flowers and siliques. This corresponds to the expression constructs LJK138, LJK139, LJK141, LJK142, LJK143 and LJK144 in table 5.

RENA molecules with SEQ ID NO9, NO10, NO11, NO12, NO13, NO14 and NO15 and a RENA-less control under control of the seed specific p-AtPXR promoter were selected for determining enhanced reliability of gene expression in transgenic oilseed rape siliques. This corresponds to the expression constructs LJK148, LJK156, LJK157, LJK158, LJK159, LJK160 and LJK161 in table 5.

3.2 Generation of Transgenic Rapeseed Plants (Amended Protocol According to Moloney et al., 1992, Plant Cell Reports, 8: 238-242).

In preparation for the generation of transgenic rapeseed plants, the binary vectors were transformed into Agrobacterium tumefaciens C58C1:pGV2260 (Deblaere et al., 1985, Nucl. Acids. Res. 13: 4777-4788). A 1:50 dilution of an overnight culture of Agrobacteria harboring the respective binary construct was grown in Murashige-Skoog Medium (Murashige and Skoog, 1962, Physiol. Plant 15, 473) supplemented with 3% saccharose (3MS-Medium). For the transformation of rapeseed plants, petioles or hypocotyledons of sterile plants were incubated with a 1:50 Agrobacterium solution for 5-10 minutes followed by a three-day co-incubation in darkness at 25° C. on 3 MS-Medium supplemented with 0.8% bacto-agar. After three days, the explants were transferred to MS-Medium containing 500 mg/l Claforan (Cefotaxime-Sodium), 100 nM Imazetapyr, 20 microM Benzylaminopurin (BAP) and 1.6 g/l Glucose in a 16 h light/8 h darkness light regime, which was repeated in weekly periods. Growing shoots were transferred to MS-Medium containing 2% saccharose, 250 mg/l Claforan and 0.8% Bacto-agar. After 3 weeks, the growth hormone 2-Indolbutyl acid was added to the medium to promote root formation. Shoots were transferred to soil following root development, grown for two weeks in a growth chamber and grown to maturity in greenhouse conditions.

3.3 Plant Analysis

Tissue samples were collected from the generated transgenic plants from leaves, flowers and siliques, stored in a freezer at −80° C. subjected to a Luciferase reporter gene assay (amended protocol after Ow et al., 1986). After grinding the frozen tissue samples were resuspended in 800 microl of buffer I (0.1 M Phosphate buffer pH7.8, 1 mM DTT (Sigma Aldrich, St. Louis, Mo., USA), 0.05% Tween 20 (Sigma Aldrich, St. Louis, Mo., USA)) followed by centrifugation at 10 000 g for 10 min. 75 microl of the aqueous supernatant were transferred to 96-well plates. After addition of 25 microl of buffer II (80 mM glycine-glycyl (Carl Roth, Karlsruhe, Germany), 40 mM MgSO₄ (Duchefa, Haarlem, The Netherlands), 60 mM ATP (Sigma Aldrich, St. Louis, Mo., USA), pH 7.8) and D-Luciferin to a final concentration of 0.5 mM (Cat No: L-8220, BioSynth, Staad, Switzerland), luminescence was recorded in a MicroLumat Plus LB96V (Berthold Technologies, Bad Wildbad, Germany) yielding the unit relative light unit RLU per minute (RLU/min). In order to normalize the luciferase activity between samples, the protein concentration was determined in the aqueous supernatant in parallel to the luciferase activity (adapted from Bradford, 1976, Anal. Biochem. 72, 248). 5 microl of the aqueous cell extract in buffer I were mixed with 250 microl of Bradford reagent (Sigma Aldrich, St. Louis, Mo., USA), incubated for 10 min at room temperature. Absorption was determined at 595 nm in a plate reader (Thermo Electron Corporation, Multiskan Ascent 354). The total protein amounts in the samples were calculated with a previously generated standard concentration curve. Values resulting from a ratio of RLU/min and mg protein/ml sample were averaged for transgenic plants harboring identical constructs and coefficient of variation was calculated to assess the impact of RENA molecule presence over RENA-less reporter gene constructs.

3.4 RENA Sequences Mediate Strongly Enhanced Reliability of Gene Expression in Oilseed Rape Plants

For assessing the potential of enhancing reliability of gene expression of selected RENA molecules (example 3.1) in oilseed rape plants, leafs, flowers and siliques harboring seeds of plants having identical developmental stages and which were grown under equal growth conditions were collected. The samples were taken from individual transgenic oilseed rape plant lines harboring either a promoter-only reporter gene construct or Luciferase reporter gene constructs containing a RENA (example 3.1). For seed-specific expression analysis 10 seeds were collected from each transgenic event, processed and analyzed for Luciferase activity as described above (Example 3.3).

RENA molecules with SEQ ID NO5, NO6, NO7, NO8 and NO16 under control of the p-AtNit1 promoter showed a reduced coefficient of variation and a reduced fraction of low expressers in B. napus leaves (table 7), and B. napus flowers (table 8) compared to a RENA-less control (LJK138).

RENA molecules with SEQ ID NO9, NO10, NO11, NO12, NO13, NO14 and NO15 under control of the seed specific p-AtPXR promoter showed a reduced coefficient of variation and a reduced fraction of low expressers in B. napus immature seeds (table 9) compared to a RENA-less control (LJK148).

TABLE 7
Coefficient of variation and fraction of low expressers (calculated as
the fraction of a population below 40% of mean expression
level of that population); OSR leaves; p-AtNit1

| | Coefficient of | fraction low expressers [%] |
|---|---|---|
| construct | variation | (<40% mean) |
| LJK138 | 0.91 | 21.62 |
| LJK139 | 0.58 | 8.57 |
| LJK141 | 0.79 | 13.16 |
| LJK142 | 0.49 | 10.53 |
| LJK143 | 0.80 | 8.57 |
| LJK144 | 0.71 | 7.69 |

TABLE 8
Coefficient of variation and fraction of low expressers (calculated as
the fraction of a population below 40% of mean expression
level of that population); OSR flowers; p-AtNit1

| | coefficient of | fraction low expressers |
|---|---|---|
| construct | variation | [%] (<40% mean) |
| LJK138 | 1.46 | 45.71 |
| LJK139 | 0.86 | 20.00 |
| LJK141 | 0.76 | 8.57 |
| LJK143 | 0.52 | 6.67 |
| LJK144 | 0.66 | 8.70 |

TABLE 9
Coefficient of variation and fraction of low expressers (calculated as
the fraction of a population below 40% of mean expression
level of that population); Immature OSR seeds; p-AtPXR

| | coefficient of | fraction low expressers |
|---|---|---|
| construct | variation | <40% of mean [%] |
| LJK148 | 1.22 | 42 |
| LJK156 | 0.66 | 21 |
| LJK157 | 0.89 | 26 |
| LJK158 | 0.65 | 17 |
| LJK159 | 0.97 | 20 |
| LJK160 | 0.80 | 30 |
| LJK161 | 0.83 | 36 |
| LJK162 | 0.59 | 15 |

3.5 Test of RENA Sequences SEQ ID NO1 to NO4 in Rapeseed

RENA sequences with SEQ ID NO1 to 4 under control of the p-AtNit1 promoter (LJH60-63) are tested analogously in rapeseed plants. Significantly enhanced reliability of gene expression is measured in leaves, flowers and siliques.

### Example 4

### Analysis of Enhanced Reliability of Gene Expression in Soybean Plants

This example illustrates that RENA molecules can be used in a wide array of plant species and across species borders from different plant families to enhance reliability of gene expression in all tissues compared to a RENA-less promoter-only approach.

RENA sequence molecules with SEQ ID NO5, NO6, NO7, NO8 and NO16 and a RENA-less control under control of the p-AtNit1 promoter were selected for determining enhanced reliability of gene expression in transgenic soybean leaves and embryos. Plant expression vectors LJK138, LJK139, LJK141, LJK142, LJK143 and LJK144 (table 5) were used for stable soybean transformation.

RENA molecules with SEQ ID NO9, NO10, NO11, NO12, NO13, NO14 and NO15 and a RENA-less control under control of the seed specific p-AtPXR promoter were selected for determining enhanced reliability of gene expression in transgenic soybean embryos. This corresponds to the expression constructs LJK148, LJK156, LJK157, LJK158, LJK159, LJK160 and LJK161 in table 5.

4.1 Generation of Transgenic Soybean Plants (Amended Protocol According to WO2005/121345; Olhoft et al., 2007).

Soybean seed germination, propagation, A. rhizogenes and axillary meristem explant preparation, and inoculations were done as previously described (WO2005/121345; Olhoft et al., 2007) with the exception that the constructs LJK138, LJK139, LJK141, LJK142, LJK143, LJK144, LJK148, LJK156, LJK157, LJK158, LJK159, LJK160 and LJK161 each contained a mutated AHAS gene driven by the parsley ubiquitin promoter PcUbi4-2, mediating tolerance to imidazolinone herbicides for selection.

4.2 RENA Sequences Mediate Strongly Enhanced Reliability of Gene Expression in Soybean Plants

Tissue samples were collected from the generated transgenic plants from leaves and seeds. The tissue samples were processed and analyzed as described above (cp. example 3.3)

In comparison to the p-AtNit1 promoter-only RENA-less reporter gene construct (LJK138), the five tested RENA molecules LJK139, LJK141, LJK142, LJK143 and LJK144 all mediated enhanced reliability of gene expression in leaves (table 10) and 12 mm embryos (table 11) measured as a reduced coefficient of variation and a reduced fraction of low expressers in the transgenic population.

In comparison to the seed specific p-AtPXR promoter-only RENA-less reporter gene construct (LJK148), the six tested RENA molecules LJK156, LJK157, LJK158, LJK159, LJK160 and LJK161 all mediated enhanced reliability of gene expression in 12 mm embryos (table 12) measured as a reduced coefficient of variation and a reduced fraction of low expressers in the transgenic population.

TABLE 10
Coefficient of variation and fraction of low expressers (calculated as
the fraction of a population below 40% of mean expression
level of that population); Soybean; leaves; p-AtNit1

| Construct | coefficient of variation | fraction low expressers (<40% mean) |
|---|---|---|
| LJK 138 | 0.92 | 16.67 |
| LJK 139 | 0.34 | 0.00 |
| LJK 141 | 0.57 | 9.09 |
| LJK 142 | 0.52 | 0.00 |
| LJK 143 | 0.81 | 8.33 |
| LJK 144 | 0.86 | 8.33 |

TABLE 11
Coefficient of variation and fraction of low expressers (calculated as
the fraction of a population below 40% of mean expression
level of that population); Soybean; 12 mm embryo; p-AtNit1

| Construct | coefficient of variation | fraction low expressers (<40% mean) |
|---|---|---|
| LJK 138 | 1.33 | 50 |
| LJK 139 | 0.65 | 40 |
| LJK 141 | 0.67 | 15 |
| LJK 142 | 0.96 | 30 |
| LJK 143 | 0.32 | 25 |
| LJK 144 | 0.48 | 0 |

TABLE 12
Coefficient of variation and fraction of low expressers (calculated as
the fraction of a population below 40% of mean expression
level of that population); Soybean; 12 mm embryo; p-AtPXR

| Construct | coefficient of variation | fraction low expressers (<40% mean) |
|---|---|---|
| LJK 148 | 1.41 | 45 |
| LJK 156 | 0.74 | 15 |
| LJK 157 | 0.54 | 15 |
| LJK 158 | 0.50 | 10 |
| LJK 159 | 0.72 | 25 |
| LJK 160 | 0.78 | 15 |
| LJK 161 | 0.63 | 20 |

4.3 Test of RENA Sequences SEQ ID NO1 to 4 in Soybean Plants

RENA sequences with SEQ ID NO1 to 4 under control of the p-AtNit1 promoter (LJH60-63) are tested analogously in soybean plants. Significantly enhanced reliability of gene expression is measured in leaves and embryos.

### Example 5

### Analysis of RENA Activity in Monocotyledonous Plants

This example describes the analysis of RENA sequences with SEQ ID NO1-NO16 in monocotyledonous plants.

5.1 Vector Construction

For analyzing RENA sequences with SEQ ID NO1-NO16 in monocotyledonous plants, two different pUC-based expression vectors are used. The first expression vector harbors an expression cassette composed of the RENA-less, constitutive monocotyledonous promoter p-Ubi from Z. mais combined with a coding sequence of the beta-Glucuronidase (GUS) gene followed by the nopaline synthase (NOS) transcriptional terminator. The second expression vector harbors an expression cassette composed of the RENA-less, seed specific monocotyledonous promoter p-KG86 from Z. mais combined with a coding sequence of the beta-Glucuronidase (GUS) gene followed by the nopaline synthase (NOS) transcriptional terminator.

Genomic DNA is extracted from A. thaliana green tissue using the Qiagen DNeasy Plant Mini Kit (Qiagen, Hilden, Germany). Genomic DNA fragments containing RENA molecules are isolated by conventional polymerase chain reaction (PCR). Primers are designed on the basis of the A. thaliana genome sequence with a multitude of RENA candidates. The reaction comprises 12 sets of primers (Table 13) and follows the protocol outlined by Phusion High Fidelity DNA Polymerase (Cat No F-540L, New England Biolabs, Ipswich, Mass., USA) using the following primers:

TABLE 13
Primer sequences

| | | SEQ ID | PCR yielding |
|---|---|---|---|
| Primer name | Sequence | NO | SEQ ID NO |
| RENA5_forII | tttttggcgcgccttagatctcgtgccgtcgtgcga | 49 | 5 |
| RENA5_revII | tttttggcgcgcctttgatcaagcctgttcaca | 50 | |
| RENA6_forII | tttttggcgcgcctttctctcgttctcatctttctctct | 51 | 6 |
| RENA6_revII | taataggcgcgcctttgtcaaacttttgattgtcacct | 52 | |
| RENA7_forII | aaaaaggcgcgcctcatcagatcttcaaaaccccaa | 53 | 7 |
| RENA7_revII | aaaaaggcgcgcctgatttgagggtagtactaaccgggaa | 54 | |
| RENA8_forII | tatatggcgcgccaaatcgttctttcaaatctctca | 55 | 8 |
| RENA8_revII | ttataggcgcgcctctgtaattcacaaaaaactgaga | 56 | |
| RENA9_forII | aataatggcgcgcctggtgcttaaacactctggtgagt | 57 | 9 |
| RENA9_revII | aataatggcgcgcctttgacctacaaaatcaaagcagtca | 58 | |
| RENA10_forII | ttttttggcgcgccagttctttgctttcgaagttgc | 59 | 10 |
| RENA10_revII | ttttttggcgcgcctactacgtactgttttcaattct | 60 | |
| RENA11_forII | tatataggcgcgccagggtttcgtttttgtttca | 61 | 11 |
| RENA11_revII | tatataggcgcgccttatctcctgctcaaagaaacca | 62 | |
| RENA12_forII | tatataggcgcgccactgtttaagcttcactgtct | 63 | 12 |
| RENA12_revII | tatataggcgcgcctttcttctaaagctgaaagt | 64 | |
| RENA13_forII | ttttttggcgcgcctttcacgatttggaatttga | 65 | 13 |
| RENA13_revII | ttttttggcgcgcctctacaacattaaaacgaccatta | 66 | |
| RENA14_forII | aataaaggcgcgccgtccagaattttctccattga | 67 | 14 |
| RENA14_revII | aataaaggcgcgcctcttcactatccaaagctctca | 68 | |
| RENA15_forII | ttttatggcgcgcctagcttaatctcagattcgaatcgt | 69 | 15 |
| RENA15_revII | ttttatggcgcgcctagtatctacataccaatcatacaaatg | 70 | |
| RENA16_forII | ttttttggcgcgccttaagaaatcctctcttctcct | 71 | 16 |
| RENA16_revII | ttttttggcgcgccctgcacatacataacatatca | 72 | |

Amplification during the PCR and purification of the amplification products is carried out as detailed above (example 1.2). Following a DNA restriction digest with AscI (10 U/microl) restriction endonuclease, the digested products are purified with the Qiagen Gel Extraction Kit (Qiagen, Hilden, Germany).

RENA PCR fragments (see above) are cloned separately upstream of the beta-Glucuronidase coding sequence using AscI restriction sites. The reaction yields one binary vector with the p-Ubi promoter, the beta-Glucuronidase coding sequence c-GUS and the t-nos terminator and one binary vector with the p-KG86 promoter, the beta-Glucuronidase coding sequence c-GUS and the t-nos terminator

In addition the reaction yields five vectors harboring SEQ ID NO5-8 and NO16 under the control of the p-Ubi promoter, immediately upstream of the beta-Glucuronidase coding sequence (Table 14), for which the combination with SEQ ID NO16 is given exemplary (SEQ ID NO82). Except for varying SEQ ID NO5-NO8, the nucleotide sequence is identical in the vectors (Table 14). The resulting vectors are summarized in table 14, with promoter molecules having the prefix p-, coding sequences having the prefix c-, and terminator molecules having the prefix t-.

In addition the reaction yields seven vectors harboring SEQ ID NO9-NO15 under the control of the p-KG86 promoter, immediately upstream of the beta-Glucuronidase coding sequence (Table 14), for which the combination with SEQ ID NO9 is given exemplary (SEQ ID NO81). Except for varying SEQ ID NO10-NO15, the nucleotide sequence is identical in the vectors (Table 14). The resulting vectors are summarized in table 14, with promoter molecules having the prefix p-, coding sequences having the prefix c-, and terminator molecules having the prefix t-.

TABLE 14
Plant expression vectors

| plant | | |
|---|---|---|
| expression | Composition of the expression cassette | SEQ ID |
| vector | Promoter::SEQ ID NO::reporter gene::terminator | NO |
| RTP2940 | p-Ubi::—::c-GUS::t-nos | |
| LJK361 | p-Ubi::SEQ ID NO16::c-GUS::t-nos | 82 |
| LJK362 | p-Ubi::SEQ ID NO5::c-GUS::t-nos | |
| LJK363 | p-Ubi::SEQ ID NO6::c-GUS::t-nos | |
| LJK364 | p-Ubi::SEQ ID NO7::c-GUS::t-nos | |
| LJK365 | p-Ubi::SEQ ID NO8::c-GUS::t-nos | |
| RTP2933 | p-KG86::—::c-GUS::t-nos | |
| LJK351 | p-KG86::SEQ ID NO9::c-GUS::t-nos | 81 |
| LJK352 | p-KG86::SEQ ID NO10::c-GUS::t-nos | |
| LJK353 | p-KG86::SEQ ID NO11::c-GUS::t-nos | |
| LJK354 | p-KG86::SEQ ID NO12::c-GUS::t-nos | |
| LJK355 | p-KG86::SEQ ID NO13::c-GUS::t-nos | |
| LJK356 | p-KG86::SEQ ID NO14::c-GUS::t-nos | |
| LJK357 | p-KG86::SEQ ID NO15::c-GUS::t-nos | |

The resulting vectors are used to analyze RENA molecules in experiments outlined below (Example 5.2).

5.2 Analysis of RENA Molecules Enhancing Reliability of Gene Expression in Monocotyledonous Plant Tissues

These experiments are performed by bombardment of monocotyledonous plant tissues or culture cells (Example 5.2.1), by PEG-mediated (or similar methodology) introduction of DNA to plant protoplasts (Example 5.2.2), or by Agrobacterium-mediated transformation (Example 5.3.3). The target tissue for these experiments can be plant tissues (e.g. leaf tissue), cultured plant cells (e.g. maize Black Mexican Sweetcorn (BMS), or plant embryos for Agrobacterium protocols.

5.2.1 Transient Assay Using Micro Projectile Bombardment

The plasmid constructs are isolated using Qiagen plasmid kit (cat#12143). DNA is precipitated onto 0.6 micrometer gold particles (Bio-Rad cat#165-2262) according to the protocol described by Sanford et al. (1993) (Optimizing the biolistic process for different biological applications. Methods in Enzymology, 217: 483-509) and accelerated onto target tissues (e.g. two week old maize leaves, BMS cultured cells, etc.) using a PDS-1000/He system device (Bio-Rad). All DNA precipitation and bombardment steps are performed under sterile conditions at room temperature. Black Mexican Sweet corn (BMS) suspension cultured cells are propagated in BMS cell culture liquid medium [Murashige and Skoog (MS) salts (4.3 g/L), 3% (w/v) sucrose, myoinositol (100 mg/L), 3 mg/L 2.4-dichlorophenoxyacetic acid (2,4-D), casein hydrolysate (1 g/L), thiamine (10 mg/L) and L-proline (1.15 g/L), pH 5.8]. Every week 10 mL of a culture of stationary cells are transferred to 40 mL of fresh medium and cultured on a rotary shaker operated at 110 rpm at 27° C. in a 250 mL flask.

60 mg of gold particles in a siliconized Eppendorf tube are resuspended in 100% ethanol followed by centrifugation for 30 seconds. The pellet is rinsed once in 100% ethanol and twice in sterile water with centrifugation after each wash. The pellet is finally resuspended in 1 mL sterile 50% glycerol. The gold suspension is then divided into 50 microL aliquots and stored at 4° C. The following reagents are added to one aliquot: 5 microL of 1 microg/microL total DNA, 50 microL 2.5 M CaCl₂, 20 microL 0.1 M spermidine, free base. The DNA solution is vortexed for 1 minute and placed at −80° C. for 3 min followed by centrifugation for 10 seconds. The supernatant is removed. The pellet is carefully resuspended in 1 mL 100% ethanol by flicking the tube followed by centrifugation for 10 seconds. The supernatant is removed and the pellet is carefully resuspended in 50 microL of 100% ethanol and placed at −80° C. until used (30 min to 4 hr prior to bombardment). If gold aggregates are visible in the solution the tubes are sonicated for one second in a water bath sonicator just prior to use.

For bombardment, two-week-old maize leaves are cut into pieces approximately 1 cm in length and placed ad-axial side up on osmotic induction medium M-N-6-702 [N6 salts (3.96 g/L), 3% (w/v) sucrose, 1.5 mg/L 2,4-dichlorophenoxyacetic acid (2,4-D), casein hydrolysate (100 mg/L), and L-proline (2.9 g/L), MS vitamin stock solution (1 mL/L), 0.2 M mannitol, 0.2 M sorbitol, pH 5.8]. The pieces are incubated for 1-2 hours.

In the case of BMS cultured cells, one-week-old suspension cells are pelleted at 1000 g in a Beckman/Coulter Avanti J25 centrifuge and the supernatant is discarded. Cells are placed onto round ash-free No 42 Whatman filters as a 1/16 inch thick layer using a spatula. The filter papers holding the plant materials are placed on osmotic induction media at 27° C. in darkness for 1-2 hours prior to bombardment. Just before bombardment the filters are removed from the medium and placed onto on a stack of sterile filter paper to allow the calli surface to partially dry.

Each plate is shot with 6 microL of gold-DNA solution twice, at 1,800 psi for the leaf materials and at 1,100 psi for the BMS cultured cells. To keep the position of plant materials, a sterilized wire mesh screen is laid on top of the sample. Following bombardment, the filters holding the samples are transferred onto M-N6-702 medium lacking mannitol and sorbitol and incubated for 2 days in darkness at 27° C. prior to transient assays.

The transient transformation via micro projectile bombardment of other monocotyledonous plants are carried out using, for example, a technique described in Wang et al., 1988 (Transient expression of foreign genes in rice, wheat and soybean cells following particle bombardment. Plant Molecular Biology, 11 (4), 433-439), Christou, 1997 (Rice transformation: bombardment. Plant Mol Biol. 35 (1-2)).

Expression levels of the expressed genes in the constructs described above (example 5.1) are determined by GUS staining, quantification of luminescence/fluorescence, RT-PCR and protein abundance (detection by specific antibodies) using the protocols in the art. GUS staining is done by incubating the plant materials in GUS solution [100 mM NaHPO4, 10 mM EDTA, 0.05% Triton X100, 0.025% X-Gluc solution (5-bromo-4-chloro-3-indolyl-beta-D-glucuronic acid dissolved in DMSO), 10% methanol, pH 7.0] at 37° C. for 16-24 hours. Plant tissues are vacuum-infiltrated 2 times for 15 minutes to aid even staining. Analyses of luciferase activities are performed as described above (example 2 and 3.3).

In comparison to the constitutive p-Ubi promoter-only RENA-less reporter gene construct, the constructs carrying RENA molecules SEQ ID NO5-NO8 and NO16 under control of the p-Ubi promoter all mediate strongly enhanced reliability of gene expression in these assays.

In comparison to the seed-specific p-KG86 promoter-only RENA-less reporter gene construct, the constructs carrying RENA molecules SEQ ID NO9-NO15 under control of the p-KG86 promoter all mediate strongly enhanced reliability of gene expression in these assays.

5.2.2 Transient Assay Using Protoplasts

Isolation of protoplasts is conducted by following the protocol developed by Sheen (1990) (Metabolic Repression of Transcription in Higher Plants. The Plant Cell 2 (10), 1027-1038). Maize seedlings are kept in the dark at 25° C. for 10 days and illuminated for 20 hours before protoplast preparation. The middle part of the leaves are cut to 0.5 mm strips (about 6 cm in length) and incubated in an enzyme solution containing 1% (w/v) cellulose RS, 0.1% (w/v) macerozyme R10 (both from Yakult Honsha, Nishinomiya, Japan), 0.6 M mannitol, 10 mM MES (pH 5.7), 1 mM CaCl₂, 1 mM MgCl₂, 10 mM beta-mercaptoethanol, and 0.1% BSA (w/v) for 3 hr at 23° C. followed by gentle shaking at 80 rpm for 10 min to release protoplasts. Protoplasts are collected by centrifugation at 100×g for 2 min, washed once in cold 0.6 M mannitol solution, centrifuged, and resuspended in cold 0.6 M mannitol (2×10⁶/mL).

A total of 50 microg plasmid DNA in a total volume of 100 microL sterile water is added into 0.5 mL of a suspension of maize protoplasts (1×10⁶ cells/mL) and mixed gently. 0.5 mL PEG solution (40% PEG 4,000, 100 mM CaNO₃, 0.5 mannitol) is added and pre-warmed at 70° C. with gentle shaking followed by addition of 4.5 mL MM solution (0.6 M mannitol, 15 mM MgCl₂, and 0.1% MES). This mixture is incubated for 15 minutes at room temperature. The protoplasts are washed twice by pelleting at 600 rpm for 5 min and resuspending in 1.0 mL of MMB solution [0.6 M mannitol, 4 mM Mes (pH 5.7), and brome mosaic virus (BMV) salts (optional)] and incubated in the dark at 25° C. for 48 hr. After the final wash step, the protoplasts are collected in 3 mL MMB medium, and incubated in the dark at 25° C. for 48 hr.

The transient transformation of protoplasts of other monocotyledonous plants are carried out using, for example, a technique described in Hodges et al., 1991 (Transformation and regeneration of rice protoplasts. Biotechnology in agriculture No. 6, Rice Biotechnology. International Rice Research Institute, ISBN: 0-85198-712-5) or Lee et al., 1990 (Transient gene expression in wheat (Triticum aestivum) protoplasts. Biotechnology in agriculture and forestry 13—Wheat. Springer Verlag, ISBN-10: 3540518096).

Expression levels of the expressed genes in the constructs described above (Example 5.1) are determined by GUS staining, quantification of luminescence/fluorescence, RT-PCR or protein abundance (detection by specific antibodies) using the protocols in the art. GUS staining is done by incubating the plant materials in GUS solution [100 mM NaHPO₄, 10 mM EDTA, 0.05% Triton X100, 0.025% X-Gluc solution (5-bromo-4-chloro-3-indolyl-beta-D-glucuronic acid dissolved in DMSO), 10% methanol, pH 7.0] at 37° C. for 16-24 hours. Analyses of luciferase activities are performed as described above (Example 2 and 3.3).

In comparison to the constitutive p-Ubi promoter-only RENA-less reporter gene construct, the constructs carrying RENA molecules SEQ ID NO5-NO8 and NO16 under control of the p-Ubi promoter all mediate strongly enhanced reliability of gene expression in these assays.

In comparison to the seed-specific p-KG86 promoter-only RENA-less reporter gene construct, the constructs carrying RENA molecules SEQ ID NO9-NO15 under control of the p-KG86 promoter all mediate strongly enhanced reliability of gene expression in these assays.

5.2.3 Transformation and Regeneration of Monocotyledonous Crop Plants

The Agrobacterium-mediated plant transformation using standard transformation and regeneration techniques may also be carried out for the purposes of transforming crop plants (Gelvin and Schilperoort, 1995, Plant Molecular Biology Manual, 2nd Edition, Dordrecht: Kluwer Academic Publ. ISBN 0-7923-2731-4; Glick and Thompson (1993) Methods in Plant Molecular Biology and Biotechnology, Boca Raton: CRC Press, ISBN 0-8493-5164-2). The transformation of maize or other monocotyledonous plants can be carried out using, for example, a technique described in U.S. Pat. No. 5,591,616. The transformation of plants using particle bombardment, polyethylene glycol-mediated DNA uptake or via the silicon carbonate fiber technique is described, for example, by Freeling & Walbot (1993) “The maize handbook” ISBN 3-540-97826-7, Springer Verlag N.Y.).

Expression levels of the expressed genes in the constructs described above (Example 5.1) are determined by GUS staining, quantification of luminescence or fluorescence, RT-PCR, protein abundance (detection by specific antibodies) using the protocols in the art. GUS staining is done by incubating the plant materials in GUS solution [100 mM NaHPO₄, 10 mM EDTA, 0.05% Triton X100, 0.025% X-Gluc solution (5-bromo-4-chloro-3-indolyl-beta-D-glucuronic acid dissolved in DMSO), 10% methanol, pH 7.0] at 37° C. for 16-24 hours. Plant tissues are vacuum-infiltrated 2 times for 15 minutes to aid even staining. Analyses of luciferase activities are performed as described above (Examples 2 and 3.3).

In comparison to the constitutive p-Ubi promoter-only RENA-less reporter gene construct, the constructs carrying RENA molecules SEQ ID NO5-NO8 and NO16 under control of the p-Ubi promoter all mediate strongly enhanced reliability of gene expression in these assays.

In comparison to the seed-specific p-KG86 promoter-only RENA-less reporter gene construct, the constructs carrying RENA molecules SEQ ID NO9-NO15 under control of the p-KG86 promoter all mediate strongly enhanced reliability of gene expression in these assays.

5.2.4 Test of RENA Sequences SEQ ID NO1 to 4 in Monocotyledonous Plants

RENA sequences with SEQ ID NO1 to NO4 under control of the p-AtNit1 promoter are tested analogously (cp. Examples 5.2.1; 5.2.2 and 5.2.3) in monocotyledonous plants. Significantly enhanced reliability of gene expression is measured in these assays compared to RENA-less control constructs.

### Example 6

### Quantitative Analysis of RENA Activity in Corn Plants

This example describes the analysis of RENA sequences with SEQ ID NO5, NO9, NO10 and NO16 in corn plants.

6.1 Vector Construction

For analyzing RENA sequences with SEQ ID NO5 and NO16 in monocotyledonous plants quantitatively, a pUC-based expression vector harboring an expression cassette composed of the RENA-less, constitutive monocotyledonous promoter p-Ubi from Z. mais was combined with a coding sequence of the firefly luciferase (LUC) gene (Promega, Madison, Wis., USA) followed by the nopaline synthase (NOS) transcriptional terminator.

For analyzing RENA sequences with SEQ ID NO9 and NO10 in monocotyledonous plants quantitatively, a pUC-based expression vector harboring an expression cassette composed of the RENA-less, seed specific monocotyledonous promoter p-KG86 from Z. mais was combined with a coding sequence of the firefly luciferase (LUC) gene (Promega, Madison, Wis., USA) followed by the nopaline synthase (NOS) transcriptional terminator.

Genomic DNA was extracted from A. thaliana green tissue using the Qiagen DNeasy Plant Mini Kit (Qiagen, Hilden, Germany). Genomic DNA fragments containing RENA molecules were isolated by conventional polymerase chain reaction (PCR). Primers were designed on the basis of the A. thaliana genome sequence with a multitude of RENA candidates. The reaction comprised 4 sets of primers (Table 15) and followed the protocol outlined by Phusion High Fidelity DNA Polymerase (Cat No F-540L, New England Biolabs, Ipswich, Mass., USA) using the following primers:

TABLE 15
Primer sequences

| | | SEQ | PCR |
|---|---|---|---|
| | | ID | yielding |
| Primer name | Sequence | NO | SEQ ID NO |
| RENA5_forIII | atatacgcgtttagatctcgtgccgtcg | 86 | 5 |
| RENA5_revIII | atatggcgcgcctttgatcaagcctgttcaca | 87 | |
| RENA16_forIII | atatacgcgtttaagaaatcctctcttctcctc | 88 | 16 |
| RENA16_revIII | atatggcgcgccctgcacatacataacatatcaagatc | 89 | |
| RENA9_forIII | atatacgcgtggtgcttaaacactctggtgagt | 90 | 9 |
| RENA9_revIII | atatggcgcgcctttgacctacaaaatcaaagcagtca | 91 | |
| RENA10_forIII | atatacgcgtagttctttgctttcgaagttgc | 92 | 10 |
| RENA10_revIII | atatggcgcgcctactacgtactgttttcaattct | 93 | |

Amplification during the PCR and purification of the amplification products was carried out as detailed above (example 1.2). Following a DNA restriction digest with MluI (10 U/microl) and AscI (10 U/microl) restriction endonucleases, the digested products were purified with the Qiagen Gel Extraction Kit (Qiagen, Hilden, Germany).

RENA PCR fragments (see above) were cloned separately upstream of the firefly luciferase coding sequence using AscI restriction sites. The reaction yielded one binary vector with the p-Ubi promoter, the firefly luciferase coding sequence c-LUC and the t-nos terminator and one binary vector with the p-KG86 promoter, the firefly luciferase coding sequence c-LUC and the t-nos terminator

In addition the reaction yielded two vectors harboring SEQ ID NO5 and NO16 under control of the p-Ubi promoter, immediately upstream of the firefly luciferase coding sequence (Table 16), for which the combination with SEQ ID NO16 is given exemplary (SEQ ID NO83). Except for varying SEQ ID NO5, the nucleotide sequence is identical in the vectors (Table 16).

In addition the reaction yielded two vectors harboring SEQ ID NO9 and NO10 under control of the p-KG86 promoter, immediately upstream of the firefly luciferase coding sequence (Table 16), for which the combination with SEQ ID NO9 is given exemplary (SEQ ID NO84). Except for varying SEQ ID NO10, the nucleotide sequence is identical in the vectors (Table 16).

The resulting vectors are summarized in table 16, with promoter molecules having the prefix p-, coding sequences having the prefix c-, and terminator molecules having the prefix t-.

TABLE 16
Plant expression vectors

| plant | | |
|---|---|---|
| expression | Composition of the expression cassette | SEQ ID |
| vector | Promoter::SEQ ID NO::reporter gene::terminator | NO |
| LJK309 | p-Ubi::—::c-LUC::t-nos | |
| LJK327 | p-Ubi::SEQ ID NO16::c-LUC::t-nos | 83 |
| LJK326 | p-Ubi::SEQ ID NO5::c-LUC::t-nos | |
| RTP5679 | p-KG86::—::c-LUC::t-nos | |
| RTP5683 | p-KG86::SEQ ID NO9::c-LUC::t-nos | 84 |
| RTP5684 | p-KG86::SEQ ID NO10::c-LUC::t-nos | |

The resulting vectors were used to analyze RENA molecules in experiments outlined below (Example 6.2).

6.2 Generation of Transgenic Maize Plants

Maize germination, propagation, A. tumefaciens preparation and inoculations were done as previously described (WO2006136596, US20090249514) with the exception that the constructs LJK309, LJK326, LJK327, RTP5679, RTP5683 and RTP5684 (cp. example 6.1) each contained a mutated AHAS gene driven by the corn ubiquitin promoter p-Ubi, mediating tolerance to imidazolinone herbicides for selection.

6.3 RENA Sequences Mediate Strongly Enhanced Reliability of Gene Expression in Corn Plants

Tissue samples were collected from the generated transgenic plants from leaves and kernels.

The tissue samples were processed and analyzed as described above (cp. example 3.3)

In comparison to the constitutive p-Ubi promoter-only RENA-less reporter gene construct (LJK309), the two tested RENA molecules under control of the p-Ubi promoter SEQ ID NO5 and NO16 (LJK326, LJK327) mediated strongly enhanced reliability of gene expression in leaves (Table 17), and kernels (Table 18).

In comparison to the seed-specific p-KG86 promoter-only RENA-less reporter gene construct (RTP5679), the two tested RENA molecules under control of the p-KG86 promoter SEQ ID NO9 and NO10 (RTP5683, RTP5684) mediated strongly enhanced reliability of gene expression in maize kernels (Table 19).

TABLE 17
Coefficient of variation and fraction of low expressers (calculated as
the fraction of a population below 40% of mean expression
level of that population); Maize leaves; p-Ubi

| | Coefficient of | fraction low expressers |
|---|---|---|
| construct | variation | (<40% mean) |
| LJK309 | 2.21 | 31.82 |
| LJK326 | 0.85 | 17.39 |
| LJK327 | 0.64 | 28.57 |

TABLE 18
Coefficient of variation and fraction of low expressers (calculated as
the fraction of a population below 40% of mean expression
level of that population); Maize kernels; p-Ubi

| | Coefficient of | fraction low expressers |
|---|---|---|
| construct | variation | (<40% mean) |
| LJK309 | 1.54 | 40.91 |
| LJK326 | 0.45 | 4.55 |
| LJK327 | 0.46 | 10.53 |

TABLE 19
Coefficient of variation and fraction of low expressers (calculated as
the fraction of a population below 40% of mean expression
level of that population); Maize kernels; p-KG68

| | Coefficient of | fraction low expressers |
|---|---|---|
| construct | variation | (<40% mean) |
| RTP5679 | 2.12 | 63.64 |
| RTP5683 | 0.98 | 15.79 |
| RTP5684 | 0.60 | 15.79 |

6.4 Test of RENA Sequences SEQ ID NO1 to NO4 and NO6 to NO8 and NO12 to NO15 Under Control of the p-Ubi Promoter and SEQ ID NO11 to NO15 Under Control of the p-KG86 Promoter in Corn Plants

RENA sequences with SEQ ID NO1 to NO4 and NO6 to NO8 and NO12 to NO15 under control of the p-Ubi promoter are tested analogously in corn plants. Significantly enhanced reliability of gene expression is measured in leaves and kernels.

RENA sequences with SEQ ID NO11 to NO15 under control of the p-KG86 promoter are tested analogously in corn plants. Significantly enhanced reliability of gene expression is measured in kernels.

### Example 7

### Quantitative Analysis of RENA Activity in Rice Plants

This example describes the analysis of RENA sequences with SEQ ID NO9, NO10 and NO16 in rice plants.

7.1 Vector Construction

For analyzing RENA sequences with SEQ ID NO16 in rice plants quantitatively, pENTR/B vectors LJK1 and LJK4 (compare example 1.3) were combined with a destination vector harboring the constitutive Sugarcane Bacilliform Virus promoter (p-ScBV) upstream of the recombination site using site specific recombination (LR-reaction) according to the manufacturers (Invitrogen, Carlsbad, Calif., USA) Gateway manual. The reactions yielded one binary vector with p-ScBV promoter, the firefly luciferase coding sequence c-LUC and the t-nos terminator as well as one vector harboring SEQ ID NO16 immediately upstream of the firefly luciferase coding sequence (Table 20).

For analyzing RENA sequences with SEQ ID NO9 and NO10 in rice plants quantitatively, pENTR/B vectors LJK1, LJK19 and LJK20 (compare example 1.3) were combined with a destination vector harboring the seed preferred rice prolamin RP6 promoter (p-RP6) upstream of the recombination site using site specific recombination (LR-reaction) according to the manufacturers (Invitrogen, Carlsbad, Calif., USA) Gateway manual. The reactions yielded one binary vector with p-RP6 promoter, the firefly luciferase coding sequence c-LUC and the t-nos terminator as well as 2 vector harboring SEQ ID NO9 or NO10 immediately upstream of the firefly luciferase coding sequence (Table 20), for which the sequence of the reporter gene expression cassette is given exemplary for the combination with SEQ ID NO9 (SEQ ID NO85). Except for varying SEQ ID NO10, the nucleotide sequence is identical in the vectors (Table 20).

The resulting vectors are summarized in table 20, with promoter molecules having the prefix p-, coding sequences having the prefix c-, and terminator molecules having the prefix t-.

TABLE 20
Plant expression vectors

| plant | | |
|---|---|---|
| expression | Composition of the expression cassette | SEQ ID |
| vector | Promoter::SEQ ID NO::reporter gene::terminator | NO |
| CD30963 | p-ScBV::—::c-LUC::t-nos | |
| CD30964 | p-ScBV::SEQ ID NO16::c-LUC::t-nos | |
| CD30977 | p-RP6::—::c-LUC::t-nos | |
| CD30971 | p-RP6::SEQ ID NO9::c-LUC::t-nos | 85 |
| CD30972 | p-RP6::SEQ ID NO10::c-LUC::t-nos | |

The resulting vectors were used to analyze RENA molecules in experiments outlined below (Example 7.2).

7.2 Generation of Transgenic Rice Plants

The Agrobacterium containing the respective expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl2, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the respective expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2.4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent TO rice transformants were generated for one construct. The primary transformants were transferred from a tissue culture chamber to a greenhouse. After a quantitative PCR analysis to verify copy number of the T-DNA insert, only single copy transgenic plants that exhibited tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

7.3 RENA Sequences Mediate Strongly Enhanced Reliability of Gene Expression in Rice Plants

Tissue samples were collected from the generated transgenic plants from leaves and kernels. The tissue samples were processed and analyzed as described above (cp. example 3.3)

In comparison to the constitutive p-ScBV promoter-only RENA-less reporter gene construct (CD30963), the tested RENA molecule SEQ ID NO 16 under control of the p-ScBV promoter (CD30964) mediated strongly enhanced reliability of gene expression measured as a reduced coefficient of variation and a reduced fraction of low expressers in leaves (table 21) and seeds (table 22).

In comparison to the seed preferred p-RP6 promoter-only RENA-less reporter gene construct (CD30977), the tested RENA molecules SEQ ID NO9 and NO10 under control of the p-RP6 promoter (CD30971 and CD30972) mediated strongly enhanced reliability of gene expression measured as a reduced coefficient of variation and a reduced fraction of low expressers in seeds (table 23).

TABLE 21
Coefficient of variation and fraction of low expressers (calculated as
the fraction of a population below 40% of mean expression
level of that population); Rice leaves; p-ScBV

| | | coefficient of | fraction low expressers |
|---|---|---|---|
| construct | Promoter | variation | (<40% mean) |
| CD30963 | p-ScBV | 3.09 | 71.43 |
| CD30964 | p-ScBV | 0.56 | 5.00 |

TABLE 22
Coefficient of variation and fraction of low expressers (calculated as
the fraction of a population below 40% of mean expression
level of that population); Rice seeds; p-ScBV

| | | coefficient of | fraction low expressers |
|---|---|---|---|
| construct | Promoter | variation | (<40% mean) |
| CD30963 | p-ScBV | 2.37 | 57.14 |
| CD30964 | p-ScBV | 0.47 | 5.88 |

TABLE 23
Coefficient of variation and fraction of low expressers (calculated as
the fraction of a population below 40% of mean expression
level of that population); Rice seeds; p-RP6

| | | coefficient of | fraction low expressers |
|---|---|---|---|
| construct | Promoter | variation | (<40% mean) |
| CD30977 | P-RP6 | 2.94 | 81.25 |
| CD30971 | P-RP6 | 0.53 | 16.67 |
| CD30972 | P-RP6 | 0.54 | 11.76 |

7.4 Test of RENA Sequences SEQ ID NO1 to NO8 and SEQ ID NO11 to NO15 in Rice Plants

RENA sequences with SEQ ID NO1 to NO8 under control of the p-ScBV promoter are tested analogously in rice plants. Significantly enhanced reliability of gene expression is measured in leaves, and seeds.

RENA sequences with SEQ ID NO11 to NO15 under control of the p-RP6 promoter are tested analogously in rice plants. Significantly enhanced reliability of gene expression is measured in seeds.

### Example 8

### Identification of Reliability Enhancing Nucleic Acids (RENA) from Other Species

8.1 Identification of RENAs in Publicly Available Genomes

RENA sequences are identified from publicly available genomic DNA sequences (e.g. http://www.ncbi.nlm.nih.gov/genomes/PLANTS/PlantList.html) of the following organisms: Zea mays, Oryza sativa, Brachypodium distachyon, Glycine max, Medicago truncatula, Sorghum bicolor, Arabidopsis lyrata, Manihot esculenta, Ricinus communis, Populus trichocarpa, Cucumis sativus, Prunus persica, Carica papaya, Citrus sinensis, Citrus clementina, Eucalyptus grandis, vinifera, Mimulus guttatus, Aqullega coerulea, Setaria italica, Selaginella moellendorffii, Physcomitrella patens, Chlamydomonas reinhardtii, Volvox carteri.

8.2 Isolation of RENA Sequences

Genomic DNA is extracted from green tissue of the respective organisms using the Qiagen DNeasy Plant Mini Kit (Qiagen, Hilden, Germany). Primer design, PCR amplification and purification for RENA sequences SEQ ID NO 94 to 116666 is performed analogously to the description in example 1.2.

8.3 Vector Construction

Using the Multisite Gateway System (Invitrogen, Carlsbad, Calif., USA), the promoter::RENA::reporter-gene cassettes for RENA sequences SEQ ID NO 94 to 116666 are assembled into binary constructs for plant transformation as described in example 1.3. The resulting plant transformation vectors are used in the subsequent experiments.

8.4 Analysis of RENA Sequences

8.4.1 Analysis of RENA Molecules Enhancing Reliability of Gene Expression in Stably Transgenic A. thaliana

For RENA sequences SEQ ID NO 94 to 116666 the respective transformation vectors are used to generate transgenic A. thaliana plants analogously to the description in example 2.1. Transgenic plants are then analysed (compare example 2.2). All tested RENA sequences (SEQ ID NO 94 to 116666) cause significantly enhanced reliability of luciferase reporter gene expression when coupled with the reporter gene compared to the RENA-less control in stably transgenic A. thaliana assays of leaves, flowers and siliques.

8.4.2 Analysis of RENA Molecules Enhancing Reliability of Gene Expression in Soybean Plants

RENA sequences with SEQ ID NO 94 to 116666 are tested in soybean plants (compare example 4). Significant enhancement of gene expression is measured in leaves, flowers and embryos.

8.4.3 Analysis of RENA Molecules Enhancing Reliability of Gene Expression in Monocotyledonous Plants

RENA sequences with SEQ ID NO 94 to 116666 are tested in monocotyledonous plants (compare example 5).

8.4.3.1 Transient Assays for Analysis in Monocotyledonous Plants

As described in example 5.2, transformation vectors containing RENA sequences SEQ ID NO 94 to 116666 are used to transiently transfect monocotyledonous plants. In both the microprojectile bombardment (compare example 5.2.1) and the transient protoplast assay (compare example 5.2.2) the RENA molecules mediate strongly enhanced reliability of gene expression in comparison to the constitutive p-Ubi promoter-only RENA-less reporter gene construct.

8.4.3.2 Assays for Analysis in Monocotyledonous Plants

RENA sequences with SEQ ID NO 94 to 116666 are tested in stably transgenic monocotyledonous plants (compare example 5.2.3). Significant enhanced reliability of gene expression is measured in all described assay (cp. Example 5.2.1, 5.2.2 and 5.2.3).

8.4.4 Analysis of RENA Molecules Enhancing Reliability of Gene Expression in Corn Plants

RENA sequences with SEQ ID NO 94 to 116666 are tested in corn plants (compare example 6).

Significantly enhanced reliability of gene expression is measured in leaves and kernels.

8.4.5 Analysis of RENA Molecules Enhancing Reliability of Gene Expression in Rice Plants

RENA sequences with SEQ ID NO 94 to 116666 are tested in rice plants (compare example 7). Significantly enhanced reliability of gene expression is measured in leaves and seeds.

### FIGURE LEGENDS

FIG. 1: Bar graph showing the coefficient of variation of a luciferase reporter gene activity in A. thaliana leaves.

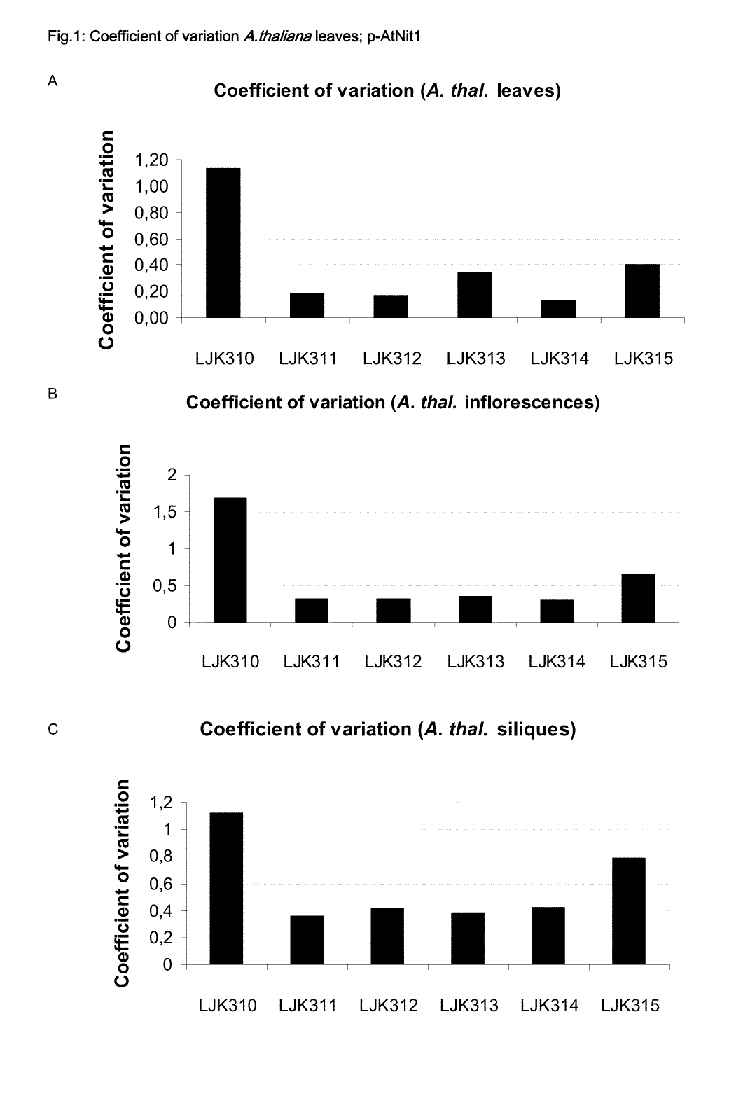

Tissues of 15 independent transgenic plants were analyzed. Relative light units (RLU) of independent transgenic oilseed rape plant lines harboring RENA-less (LJK310) or RENA-containing reporter gene constructs (LJK311-LJK315) under the control of the p-AtNit1 promoter were calculated by normalizing against the protein content of each sample. Mean as well as standard deviation were calculated. Coefficient of variation was calculated from the standard deviation divided by the mean of expression. Coefficient of variation serves as an indicator for reliability of gene expression independent from level of expression. A) leaf tissue, B) flowers, C) siliques.

1. A method for reducing the coefficient of variation of expression in a population of plants comprising the steps of:
a) providing a recombinant nucleic acid comprising at least one Reliability Enhancing Nucleic Acid (RENA) molecule selected from the group consisting of:
i) a nucleic acid molecule having a sequence selected from SEQ ID NOS: 1 to 16 or 94 to 116666;
ii) a nucleic acid molecule having a sequence with an identity of at least 80% to a sequence selected from SEQ ID NOS: 1 to 16 or 94 to 116666;
iii) a fragment of at least 100 consecutive bases of a nucleic acid molecule of i) or ii) which has reliability enhancing activity as the corresponding nucleic acid molecule having the sequence of selected from SEQ ID NOS: 1 to 16 or 94 to 116666;
iv) a nucleic acid molecule hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. to a nucleic acid molecule comprising at least 50 consecutive nucleotides of the complement of a reliability enhancing nucleic acid having a sequence selected from SEQ ID NOS: 1 to 16 or 94 to 116666; or
v) a nucleic acid molecule which is the complement or reverse complement of any of the previously mentioned nucleic acid molecules under i) to iv);
b) functionally linking the RENA molecule as defined under i) to v) to a promoter and/or a nucleic acid molecule of interest wherein at least the promoter is heterologous to the RENA molecule;
c) integrating the RENA molecule functionally linked to a promoter and/or a nucleic acid molecule of interest in the genome of plant cells, plants or parts thereof; and
d) regenerating a population of plants from the transgenic plant cells, plants or parts thereof produced in step c);
wherein the molecules as defined under ii) to v) reduce the variation of expression at by least 10% of the corresponding nucleic acid molecule having the sequence selected from SEQ ID NOS: 1 to 16 or 94 to 116666; and
wherein the population of independent primary transformant plants exhibit an at least 10% lower coefficient of variation of expression of the nucleic acid molecule of interest compared to a population of independent primary transformant plants comprising in their genome a respective recombinant nucleic acid construct not comprising the respective RENA molecule as defined in i) to v).

2. A method for producing a population of plants with reduced percentage of low-expressing plants comprising the steps of:
a) providing a recombinant nucleic acid construct comprising at least one RENA molecule selected from the group consisting of:
i) a nucleic acid molecule having a sequence selected from SEQ ID NOS: 1 to 16 or 94 to 116666;
ii) a nucleic acid molecule having a sequence with an identity of at least 80% to a sequence selected from SEQ ID NOS: 1 to 16 or 94 to 116666;
iii) a fragment of at least 100 consecutive bases of a nucleic acid molecule of i) or ii) which has reliability enhancing activity as the corresponding nucleic acid molecule having the sequence selected from SEQ ID NOS: 1 to 16 or 94 to 116666;
iv a nucleic acid molecule hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. to a nucleic acid molecule comprising at least 50 consecutive nucleotides of the complement of a reliability enhancing nucleic acid having a sequence selected from SEQ ID NOS: 1 to 16 or 94 to 116666; and
v) a nucleic acid molecule which is the complement or reverse complement of any of the previously mentioned nucleic acid molecules under i) to iv);
functionally linked to a promoter and/or a nucleic acid molecule of interest wherein at least the promoter is heterologous to the RENA molecule;
b) integrating said recombinant nucleic acid construct in the genome of plant cells, plants or parts thereof; and
c) regenerating a population of independent primary transformant plants from the transgenic plant cells, plants or parts thereof produced in step b);
wherein the population of plants has an at least 5% reduced percentage of plants exhibiting low expression of the nucleic acid of interest compared to a population of plants comprising in their genome a respective recombinant nucleic acid construct not comprising the respective RENA molecule as defined in a) i) to v).

3. A method for reducing the number of low expressing plants in a population of independent primary transformant plants, comprising the steps of
a) providing a recombinant nucleic acid construct comprising at least one RENA molecule selected from the group consisting of:
i) a nucleic acid molecule having a sequence selected from SEQ ID NOS: 1 to 16 or 94 to 116666;
ii) a nucleic acid molecule having a sequence with an identity of at least 80% to a sequence selected from SEQ ID NOS: 1 to 16 or 94 to 116666;
iii) a fragment of at least 100 consecutive bases of a nucleic acid molecule of i) or ii) which has reliability enhancing activity as the corresponding nucleic acid molecule having the sequence selected from SEQ ID NOS: 1 to 16 or 94 to 116666;
iv) a nucleic acid molecule hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. to a nucleic acid molecule comprising at least 50 consecutive nucleotides of the complement of a reliability enhancing nucleic acid having a sequence selected from SEQ ID NOS: 1 to 16 or 94 to 116666; and
v) a nucleic acid molecule which is the complement or reverse complement of any of the previously mentioned nucleic acid molecules under i) to iv);
functionally linked to a promoter and/or a nucleic acid molecule of interest wherein at least the promoter is heterologous to the RENA molecule;
b) integrating said recombinant nucleic acid construct in the genome of plant cells, plants or parts thereof; and
c) regenerating independent primary transformant plants from the transgenic plant cells, plants or parts thereof produced in step b);
wherein the number of low expressing independent primary transformant plants is reduced by at least 5% compared to a population of independent primary transformant plants comprising in their genome a respective recombinant nucleic acid not comprising the respective RENA molecule as defined in a) i) to v).

4. The methods of claim 1, wherein the RENA molecule is comprised in an expression construct or vector functionally linked to a promoter and/or a nucleic acid molecule of interest wherein at least the promoter is heterologous to the at least one RENA molecule.

5. The method of claim 1, wherein said at least one RENA molecule is located 2500 bp or less away from the transcription start site of said heterologous nucleic acid molecule of interest to which it is functionally linked.

6. The method of claim 5, wherein said at least one RENA molecule is located upstream of the translational start site of the nucleic acid molecule to which it is functionally linked.

7. The method of claim 6, wherein said at least one RENA molecule is located within a 5′UTR of the nucleic acid molecule of interest to which it is functionally linked.

8. A recombinant expression construct comprising at least one RENA molecule selected from the group consisting of:
i) a nucleic acid molecule having a sequence selected from SEQ ID NOS: 1 to 16 or 94 to 116666;
ii) a nucleic acid molecule having a sequence with an identity of at least 80% to a sequence selected from SEQ ID NOS: 1 to 16 or 94 to 116666;
iii) a fragment of at least 100 consecutive bases of a nucleic acid molecule of i) or ii) which has reliability enhancing activity as the corresponding nucleic acid molecule having the sequence selected from SEQ ID NOS: 1 to 16 or 94 to 116666;
iv) a nucleic acid molecule hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. to a nucleic acid molecule comprising at least 50 consecutive nucleotides of the complement of a reliability enhancing nucleic acid having a sequence selected from SEQ ID NOS: 1 to 16 or 94 to 116666; and
v) a nucleic acid molecule which is the complement or reverse complement of any of the previously mentioned nucleic acid molecules under i) to iv);
functionally linked to a promoter and/or a nucleic acid molecule of interest wherein at least the promoter is heterologous to the RENA molecule.

9. The method of claim 1, wherein the promoter is a constitutive promoter, a tissue-specific or tissue-preferential promoter, a developmental-specific or developmental-preferential promoter or an inducible promoter.

10. A recombinant expression vector comprising one or more recombinant expression constructs of claim 8.

11. A transgenic cell or transgenic plant or part thereof comprising:
at least one RENA molecule selected from the group consisting of:
i) a nucleic acid molecule having a sequence selected from SEQ ID NOS: 1 to 16 or 94 to 116666;
ii) a nucleic acid molecule having a sequence with an identity of at least 80% to a sequence selected from SEQ ID NOS: 1 to 16 or 94 to 116666;
iii) a fragment of at least 100 consecutive bases of a nucleic acid molecule of i) or ii) which has reliability enhancing activity as the corresponding nucleic acid molecule having the sequence selected from SEQ ID NOS: 1 to 16 or 94 to 116666;
iv) a nucleic acid molecule hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. to a nucleic acid molecule comprising at least 50 consecutive nucleotides of the complement of a reliability enhancing nucleic acid having a sequence selected from SEQ ID NOS: 1 to 16 or 94 to 116666; and
v) a nucleic acid molecule which is the complement or reverse complement of any of the previously mentioned nucleic acid molecules under i) to iv);
functionally linked to a heterologous promoter and/or a heterologous nucleic acid of interest to be expressed.

12. The transgenic cell or transgenic plant or part thereof of claim 11 selected from the group consisting of bacteria, fungi, yeasts or plants.

13. (canceled)

14. A method for the production of an agricultural product by introducing a RENA molecule selected from the group consisting of:
i) a nucleic acid molecule having a sequence selected from SEQ ID NOS: 1 to 16 or 94 to 116666;
ii) a nucleic acid molecule having a sequence with an identity of at least 80% to a sequence selected from SEQ ID NOS: 1 to 16 or 94 to 116666;
iii) a fragment of at least 100 consecutive bases of a nucleic acid molecule of i) or ii) which has reliability enhancing activity as the corresponding nucleic acid molecule having the sequence selected from SEQ ID NOS: 1 to 16 or 94 to 116666;
iv) a nucleic acid molecule hybridizing under conditions equivalent to hybridization in 7% sodium dodecyl sulfate (SDS), 0.5 M NaPO₄, 1 mM EDTA at 50° C. with washing in 2×SSC, 0.1% SDS at 50° C. to a nucleic acid molecule comprising at least 50 consecutive nucleotides of the complement of a reliability enhancing nucleic acid having a sequence selected from SEQ ID NOS: 1 to 16 or 94 to 116666; and
v) a nucleic acid molecule which is the complement or reverse complement of any of the previously mentioned nucleic acid molecules under i) to iv);
into a plant, growing the plant, harvesting and processing the plant or parts thereof.

15. The method of claim 4, wherein the RENA molecule is heterologous to both the promoter and the nucleic acid of interest.

16. The recombinant expression construct of claim 8, wherein the RENA molecule is heterologous to both the promoter and the nucleic acid of interest.

17. A transgenic cell or transgenic plant or part thereof comprising the recombinant expression construct of claim 8.

18. A transgenic cell or transgenic plant or part thereof comprising the recombinant expression vector of claim 10.

19. A method for the production of an agricultural product by introducing the recombinant expression construct of claim 8 into a plant, growing the plant, harvesting and processing the plant or parts thereof.

20. A method for the production of an agricultural product by introducing the recombinant expression vector of claim 10 into a plant, growing the plant, harvesting and processing the plant or parts thereof.
